(19) United States
(12) Patent Application Publication
(10) Pub. No.: US 2021/0323940 A1
(43) Pub. Date: Oct. 21, 2021

(54) NOVEL MOLECULE WITH ANTI-CANCER ACTIVITY

(71) Applicant: OCEANS LTD., St-Johns (CA)

(72) Inventors: Judith BOBBITT, St-Johns (CA); Ahmed ZEIN, St-Johns (CA)

(73) Assignee: OCEANS LTD., St-Johns, NL (CA)

(21) Appl. No.: 16/972,750

(22) PCT Filed: Jun. 7, 2019
(86) PCT No.: PCT/CA2019/050797

§ 371 Date: Dec. 7, 2020

Related U.S. Application Data
(60) Provisional application No. 62/681,886, filed on Jun. 7, 2018.

(51) Int. Cl. C07D 313/00; A23L 33/10; A61K 45/06; A61P 35/04

(52) U.S. Cl. CPC C07D 313/00; A23L 33/10; A61K 9/0053; A61P 35/04; A61K 45/06

(57) ABSTRACT

The present invention provides a new compound of formula (II), extracted from Chaetomorpha Cannabina seaweed. (II) The methods for extraction from the seaweed, the fractionation and isolation of the compound, the determination of the stereochemistry and its use for inhibiting the growth of cancer cells are provided.

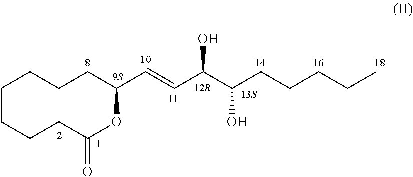 [SMILES: CCCCC[C@H](O)[C@H](O)/C=C/[C@@H]1CCCCCCCC(=O)O1]

### FIELD OF THE INVENTION

The present invention relates to a novel molecule, originally extracted from seaweeds, method of preparation and use for inhibiting the growth of cancer cells.

### BACKGROUND OF THE INVENTION

Cancer is a disease that seriously jeopardizes the health of human beings. Around the globe, about 6 million people die of cancer every year, with another 10 million seriously affected by the disease. According to the estimate of the World Health Organization, in the 21st century, cancer will become the “number one killer” of mankind.

In the several past decades, many ways of treating cancer became available, mainly including surgery, radiotherapy, chemotherapy, hormonotherapy, gene therapy, and immunotherapy, among which surgery, radiotherapy and chemotherapy have become the major means. Chemotherapy refers to treating cancer with chemical medication. It is the most rapidly expanding field in the treatment of cancer. A great number of new medicines aiming at different targets are ready for clinical application, and developments in research in mechanism of drug action and pharmacokinetics have made the clinical administration routes and means more fitting for killing tumor cells while protecting the normal tissues.

The search for natural-derived molecule for inhibiting cancer cells has led to the discovery of molecules such as Taxol or Vinblastine. Despite the utility of taxus and vinca alkaloids in the clinic, there are serious limitations to these therapies.

One major drawback when treating cancer is to achieve selectivity against this type of cancer cells. There remains a need to discover and isolate new potent compounds having selective activity against certain types of cancer cells, thereby providing highly selective anti-cancer molecules.

Against such a background, new molecules to add to the already existing armada of chemotherapeutic drugs are highly desirable.

### SUMMARY OF THE INVENTION

A main aspect intended to be addressed by the present invention is to provide a novel molecule defined by formula (II):

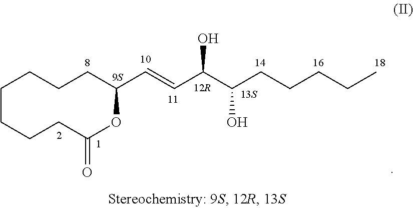 [SMILES: CCCCC[C@H](O)[C@H](O)/C=C/[C@@H]1CCCCCCCC(=O)O1]

According to a first aspect, there is provided a compound defined by formula (II) in separated, enriched or purified form.

According to a further aspect of the present invention, there is provided use of compound (II) as defined herein for inhibiting growth of cancer cells, either in vitro or in vivo, for example in a mammal.

According to a further aspect of the present invention, there is provided use of compound (II) as defined herein for the manufacture of composition for treating cancer in a mammal.

According to a further aspect, the present invention provides a composition comprising the compound as defined herein, in admixture with a physiologically acceptable excipient.

According to a further aspect, the present invention provides use of the composition as defined herein for the treatment of cancer in a mammal.

According to a further aspect, the present invention provides a method for inhibiting growth of cancer cells comprising contacting said cell with a growth-inhibiting concentration of the compound, or the composition, as defined herein.

According to a further aspect, the present invention provides a method for treating cancer in a mammal comprising administering a growth-inhibiting concentration of the composition as defined herein to the mammal.

### DETAILED DESCRIPTION OF THE INVENTION

### Description of the Figures

FIG. 1 Picture of a sample of Chaetomorpha Cannabina (CC) seaweed used for extraction.

FIG. 2. In vitro activity of different concentrations of crude extracts #1 (NC77) on the viability of seven cell lines.

FIG. 3. Fractionation and sub-fractionation strategy for the crude extract #1 of Chaetomorpha Cannabina (NC77).

FIG. 4. In vitro activity of fractions (F1-F5) from crude extract #1 (NC77) on viability of five cancer cell lines.

FIG. 5. GC chromatogram of FAME prepared from NC77 and NC77-Fr-4 fraction.

FIG. 6. ¹H-NMR profile for NC77 extract.

FIG. 7. ¹H-NMR profiles for NC77-Fr-4 fraction.

FIG. 8. HPLC comparison between two samples of NC77-Fr4 from two different harvesting dates.

FIG. 9. Flowchart of fractionation, sub-fractionation and purification of main components from NC77.

FIG. 10. UPLC-DAD/ELSD/HRMS profile of compound of formula (I).

FIG. 11. HRMS spectra of compound of formula (I).

FIG. 12. ¹H-NMR spectrum of compound of formula (I).

FIG. 13. COSY spectrum of compound of formula (I).

FIG. 14. TOCSY spectrum of compound of formula (I).

FIG. 15. HSQC spectrum of compound of formula (I).

FIG. 16. HMBC spectrum of compound of formula (I).

FIG. 17. Fragment ions observed in HRMS for compound of formula (I).

FIG. 18. ¹H-NMR spectra of NC77(2)-44-46 and its 12-S/R Mosher esters.

FIG. 19. COSY spectrum of 12-(S) Mosher ester from R (−)-MTPA-Cl.

FIG. 20. COSY spectrum of 12-(R) Mosher ester from S (+)-MTPA-Cl.

FIG. 21. The phenyl shielding effects in 12R-O-S-MTPA and 12R-O-R-MTPA esters.

FIG. 22. Proton NMR spectra of NC77(2)-44-46 and its 12,13-bis MTPA esters.

FIG. 23. COSY spectrum of 12,13-bis (S) Mosher ester prepared from R (−)-MTPA-Cl.

FIG. 24. COSY spectrum of 12,13-bis (R) Mosher ester prepared from S (+)-MTPA-Cl.

FIG. 25. HSQC spectrum of 12,13-bis (S) Mosher ester prepared from R (−)-MTPA-Cl.

FIG. 26. HSQC spectrum of 12,13-bis (R) Mosher ester prepared from S (+)-MTPA-Cl.

FIG. 27. The phenyl shielding effects in 12R,13S-bis-O-S-MTPA and 12R,13S-bis-O-R-MTPA esters.

FIG. 28. Structure of compound (II) with its defined stereochemistry.

FIG. 29. Tumour volume over the four-week treatment duration.

FIG. 30. Body weights over the four-week treatment duration.

FIG. 31. Representative photos of tumours at necropsy. A. external view of a tumour at 4 weeks in a representative non-responding animal. B. necropsy view of a tumour at 4 weeks in a representative control animal. C. necropsy view of a tumour at 4 weeks in a representative pure compound-treated animal.

FIG. 32. Tumour volumes over the four-week treatment duration.

FIG. 33. Body weights over the four-week treatment duration.

FIG. 34. Effect of DHODO on the growth (tumour volumes) of NDA-MB-231 flank tumour in J:nu mice

### ABBREVIATIONS AND DEFINITIONS

### Abbreviations

bis-AAF-R110: bis-alanyl-alanyl-phenylalanyl-rhodamine 110; CIMA: colorimetric indicative of metabolic activity; GF-AFC: Gly-Phe-7-amino-4-trifluoromethylcoumarin; HILIC: hydrophilic interaction liquid chromatography. C-18 SPE: solid phase extraction on C-18 column.

### Definitions

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

The terms “about” or “around” as used herein refers to a margin of + or −10% of the number indicated. For sake of precision, the term about when used in conjunction with, for example: 90% means 90%+/−9% i.e. from 81% to 99%. More precisely, the term about refers to + or −5% of the number indicated, where for example: 90% means 90%+/−4.5% i.e. from 86.5% to 94.5%. When used in the context of a pH, the term about means+/−0.5 pH unit.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

As used herein, the terms “disease” and “disorder” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The term “subject” or “patient” as used herein refers to an animal, preferably a mammal, and most preferably a human who is the object of treatment, observation or experiment.

“Mammal” includes humans and both domestic animals such as laboratory animals and household pets, (e.g. cats, dogs, swine, cattle, sheep, goats, horses, rabbits), and non-domestic animals such as wildlife and the like.

The term “extract” as used herein means a composition prepared by contacting solvent with seaweed biomass, produced following the procedures of the invention, which demonstrates inhibitory activity against one or more cancer cell line in vitro. In one aspect of the invention, an extract demonstrates inhibitory activity against cancer cell growth in vivo. As used herein, the term “extract” means an extract that is: crude, fractionated, sub-fractionated, separated, isolated, enriched or purified, without being limited thereto.

The term “isolated” is used herein to indicate that the compound exists in a physical milieu distinct from that in which it occurs in nature. For example, the isolated molecule may be substantially isolated (for example enriched) with respect to the complex cellular milieu in which it naturally occurs, such as in a crude extract. When the isolated molecule is enriched, the absolute level of enrichment is not critical and those skilled in the art can readily determine appropriate levels of purity according to the use to which the biomass is to be put. In some circumstances, the isolated molecule forms part of a composition (for example a more or less crude extract containing many other substances) or buffer system, which may for example contain other components. In other circumstances, the isolated molecule may be enriched to essential homogeneity, for example as determined spectrophotometrically, by NMR or by chromatography (for example LC-MS).

The term “separated” is used herein to indicate that the compound is present in diastereomer form that is enriched in one stereomer compared to another, for example, up to 100% pure diastereomer.

The molecule(s) described herein can be formulated as pharmaceutical compositions by formulation with additives such as pharmaceutically acceptable excipients, pharmaceutically acceptable carriers, and pharmaceutically acceptable vehicles.

As used herein, the term “pharmaceutically acceptable” refers to molecular entities and compositions that are physiologically tolerable and do not typically produce an allergic or similar unwanted reaction, such as gastric upset, dizziness and the like, when administered to human. Preferably, as used herein, the term “pharmaceutically acceptable” means approved by regulatory agency of the federal or state government or listed in the U.S. Pharmacopeia or other generally recognized pharmacopeia for use in animals, and more particularly in humans.

The term “carrier” refers to a diluent, adjuvant, excipient, or vehicle with which the compounds of the present invention may be administered. Sterile water or aqueous saline solutions and aqueous dextrose and glycerol solutions may be employed as carrier, particularly for injectable solutions. Suitable pharmaceutical carriers are described in “Remington's Pharmaceutical Sciences” by E. W. Martin.

The terms “molecule” and “compound” are used herein interchangeably.

As used herein, the terms “pure” or “purified” is used herein to indicate that the compound may be substantially enriched with respect to the complex cellular milieu in which it naturally occurs, such as in a crude extract. It can also be produced in synthetic manner, distinct from the isolation or extraction from its natural milieu, while still having the same molecular structure. When the molecule is purified, the absolute level of purity is not critical and those skilled in the art can readily determine appropriate levels of purity according to the use to which the biomass is to be put. In some circumstances, the isolated molecule forms part of a composition (for example a more or less crude extract containing many other substances) or buffer system, which may for example contain other components. In other circumstances, the isolated molecule may be purified to essential homogeneity, for example as determined spectrophotometrically, by NMR or by chromatography (for example LC-MS). In certain embodiments, the term “purified” means: at least 90%, for example, 90% or 91% or 92% or 93% or 94% or 95% or 96% or 97% or 98% or 99% or 99.5% or 99.6% or 99.8% or 99.9% or 100% pure.

### Detailed Description of Particular Aspects of the Invention

With the aim of providing an alternative source of anti-cancer molecules, there is provided an anti-cancer compound extracted and isolated from a solvent extract of Chaetomorpha Cannabina (CC).

### Method of Extraction and Isolation

In accordance with a particular embodiment, there is provided a method for extracting the compound (II) as defined herein, comprising the steps of:
a) mixing biomass from Chaetomorpha Cannabina (CC) seaweed with a first solvent to obtain a biomass:first solvent mixture;
b) eluting said mixture in SPE column with various concentrations of a second solvent and recovering a second solvent fraction;
c) fractionating said methanol fraction from step b) on Combiflash column with various concentrations of a third solvent mixture, and recovering a fraction containing major components.

In accordance with an alternative embodiment, the method of the invention further comprises a hexane-defatting step prior to step a).

In accordance with a particular embodiment, the method further comprises the step of: d) sub-fractionating said fraction on semi-preparative HPLC column with a fourth solvent mixture to obtain a sub-fraction enriched in compound (I).

In accordance with an optional embodiment, the method further comprises the step of: d′) enriching or purifying or separating a compound of formula (II) from said sub-fraction:

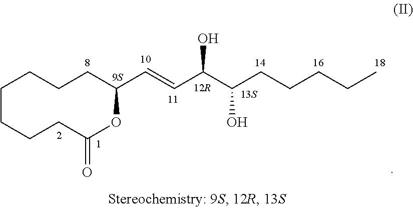 [SMILES: CCCCC[C@H](O)[C@H](O)/C=C/[C@@H]1CCCCCCCC(=O)O1]

In accordance with an alternative embodiment, the method further comprises the step of: e) drying said sub-fraction by removing solvent to obtain a dried extract enriched in the compound of formula (II).

### Solvent for Extraction

Particularly, the molecule is extracted with a first solvent. More particularly, the extract's first solvent is water or alcohol; and even more particularly: aqueous ethanol.

Particularly, the crude extract is an aqueous ethanol extract of CC. More particularly, the crude extract is a: 80%, 75%, 70%, 65%, 60%, 55%, 50%, 45%, 40%, 35%, 30%, 25% aqueous ethanol extract. Even more particularly, the crude extract is a: 80% aqueous ethanol extract. Most particularly, the crude extract is a previously hexane-defatted extract.

Particularly, the second solvent is methanol, aqueous methanol or a mixture of methanol and acetonitrile. More particularly, the extract is a C-18 second solvent fraction of the crude extract: particularly a 5%, 10%, 15%, 20%, 25%, 30%, 25%, 40%, 45%, 50%, 55%, 60%, 65%, 70%, 75%, 80%, 85%, 90%, 95% of a second solvent, being particularly: aq. MeOH, or 100% MeOH, or CH₂Cl₂:MeOH (1:1). Most particularly, the fraction is from a 30% aqueous MeOH to 100% MeOH fraction.

Particularly, the third solvent is methanol or aqueous methanol. More particularly, the extract is a flash column sub-fraction of the C-18 fraction. Particularly, the sub-fraction is about 40% MeOH sub-fraction.

### Extract

In accordance with a particular embodiment of the present invention, the bioactive molecule from the above listed seaweeds is found in a C-18 SPE MeOH fraction from an aqueous-ethanol crude extract of Chaetomorpha Cannabina (CC).

### Active Molecule

According to a particular embodiment, the invention provides a compound as defined by formula (I):

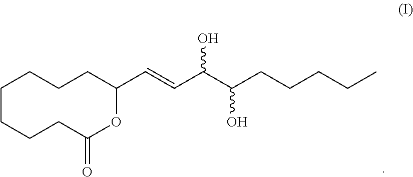 [SMILES: CCCCCC(O)C(O)/C=C/C1CCCCCCCC(=O)O1]

as a racemic mixture or diastereomer thereof.

### Stereochemistry

According to a particular embodiment, the invention provides a compound as defined by formula (II):

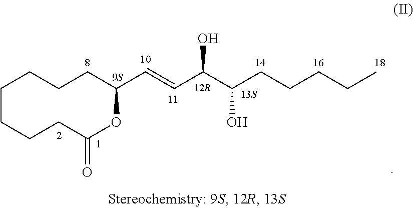 [SMILES: CCCCC[C@H](O)[C@H](O)/C=C/[C@@H]1CCCCCCCC(=O)O1]

### Composition

In accordance with a particular embodiment of the invention, there is provided a composition comprising the compound (II) as defined herein, in admixture with a physiologically acceptable excipient.

### Uses and Methods of Use

In accordance with an alternative embodiment, the present invention provides the use of the compound (I) or (II) as defined herein for inhibiting growth of cancer cells. Particularly, there is provided the use of the compound (II) as defined herein for the manufacture of composition for treating cancer in a mammal.

In accordance with an alternative embodiment of the invention, there is provided the use of the composition as defined herein for the treatment of cancer in a mammal.

In accordance with the uses as defined herein, the cancer is selected from the group consisting of: brain, lung, prostate, blood, breast and ovarian cancers. Particularly, the cancer is brain cancer. Particularly, the cancer is lung cancer. Particularly, the cancer is prostate cancer. Particularly, the cancer is leukemia (i.e. blood cancer). Particularly, the cancer is breast cancer. Particularly, the cancer is ovarian cancer.

In accordance with a particular embodiment, the present invention provides a method of inhibiting a cancer cell growth comprising contacting said cell with a growth-inhibiting concentration of the compound (II) as defined herein or the composition as defined herein.

In accordance with and method of inhibiting as defined herein, the cancer cells are selected from the group consisting of: brain, lung, prostate, blood, breast and ovaries cancer cells.

### Method of Treatment

More particularly, there is provided a method of treatment of cancer in a mammal comprising administering a growth-inhibiting concentration of the composition as defined herein to said mammal. Most particularly, the mammal is a pet animal or a human.

In accordance with the method of treatment as defined herein, the cancer is selected from the group consisting of: brain, lung, prostate, blood, breast and ovarian cancers. Particularly, the cancer is brain cancer. Particularly, the cancer is lung cancer. Particularly, the cancer is prostate cancer. Particularly, the cancer is leukemia (i.e. blood cancer). Particularly, the cancer is breast cancer. Particularly, the cancer is ovarian cancer.

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES—ANTI-CANCER ACTIVITY OF COMPOUND (I) PURIFIED FROM MARINE SEAWEED HARVESTED FROM NEWFOUNDLAND AND LABRADOR

### Example 1—Seaweed Collection and Identification

A collection program for seaweeds was established for different geographical regions of Newfoundland, Labrador and Quebec over several time periods.

The general collection procedure was as follows: seaweeds were collected from the intertidal zone by hand with knives while scuba divers collected seaweed from sub tidal zones. Samples were placed in plastic sampling bags and transported to the laboratory in coolers of seawater. Upon arrival in the laboratory, each species was washed individually to remove epiphytic and extraneous matter (sand, mussels, isopods, etc.). Samples were then checked visually to ensure they were clean. If not, remaining matter was removed by hand with further washing. Seaweeds were blotted dry, weighed to the nearest g (plant wet weight) and shredded. The shredded biomass was transferred into Erlenmeyer flasks and frozen at −60° C. until the extracts were prepared.

A representative sample of different samples of Chetomorpha cannabina were also photographed (see FIG. 1) and frozen at −20° C. for confirmation of species by Dr. Robert Hooper, a phycologist at Memorial University of Newfoundland.

### Example 2—Extract Preparation

### 2.1 Freeze-Drying

To prepare samples for extraction the seaweed was first freeze dried. Erlenmeyer flasks containing shredded seaweeds, which had been frozen at −60° C., were placed on a freeze-dryer, and lyophilized for 72-96 h at 69×10⁻³ mbar. The weight (g) of dry biomass was then recorded as plant dry weight (g). This step accounts for the differences in water content among seaweeds which may otherwise affect the solubility of bioactive components. Secondary plant metabolites are also more stable when stored in a dried form. Moreover, the large-scale extraction of dried plant biomass may cause fewer problems than extracting fresh biomass. In order to preserve thermo-labile compounds, low temperature conditions are used throughout the process of extraction.

### 2.2 Defatting of Samples

The lipid fraction of seaweed is known to vary from 1 to 5% of the algal dry matter, which can be dominated by polyunsaturated fatty acids. Brown and red seaweeds are particularly rich in long chain polyunsaturated fatty acids such as eicosapentaenoic acid (n3, C20:5), while green seaweeds may possess a level of alpha linoleic acid (n3, C18:3). Since these polyunsaturated fatty acids are extremely susceptible to oxidation, they may result in lipid oxidation products during analysis. In order to eliminate the above oxidative processes that may have an effect on the results, samples were defatted prior to extraction of compounds.

Freeze dried seaweed samples were ground into a powder and defatted by blending the powder with hexane (1:5, w/v, 5 min) in a Waring blender at ambient temperature. Defatted samples were air-dried, vacuum packed in polyethylene pouches and kept at −20° C. until extraction.

### 2.3 Crude Extraction

Different solvents or solvent systems can be used for the extraction of compounds. In general, ethanol is commonly used due to its lower toxicity compared to other solvents. Moreover, ethanol extracts have been demonstrated in many studies to have the highest antioxidant activity.

Compounds were extracted into 80% aqueous ethanol at 4° C. for 24 h. The solvent was then removed under a vacuum at 37° C. for 45 to 60 min and the resulting concentrated slurries were lyophilized for 72 to 96 h at −80° C. and 69×10⁻³ mbar using a freeze dryer. Dry extracts were weighed (Extract dry weight in g) and stored at −60° C. until preparation for screening.

Extraction yields were calculated and expressed as g of dry extract per g of dry seaweed as per Table 1.

TABLE 1
Extraction Yields

| | | | | | Yield |
|---|---|---|---|---|---|
| | | | | Exract | (g of dry |
| | | | | dry | extract/ |
| | | Date | | weight | g of dry |
| Species | Seaweed | Collection | Location | (g) | plant |
| Chaetomorpha | Green | Jul. | Rocky | 4.88 | 8.0% |
| cannibina | | 22nd, | Hr., | | |
| (extract # 1 for | | 2013 | NL | | |
| NC 77) | | | | | |
| Chaetomorpha | Green | Aug. | Mutton | 0.7 | 3.88% |
| cannibina | | 26, | Bay, | | |
| (extract #2 or | | 2014 | Quebec | | |
| NC 107) | | | | | |

### Example 3. Anti-Cancer Screening of Seaweed Crude Extracts

### 3.1 Purification Through Bioassay-Guided Fractionation

Initially, seaweed extracts were evaluated for their anti-cancer activity in in vitro models via the CIMA assay. From these results, extracts exhibiting the greatest anti-cancer potential were selected for purification via bioassay-guided fractionation.

### 3.2 Compound Preparation

Stock solutions of the extracts were prepared in dimethylsulfoxide (DMSO) at 10 mg/mL and stored in 200 μl aliquots at −20° C. until analysis. This preparation ensured that the DMSO delivered to cells in culture never exceeded 1%.

### 3.3 CIMA Assay

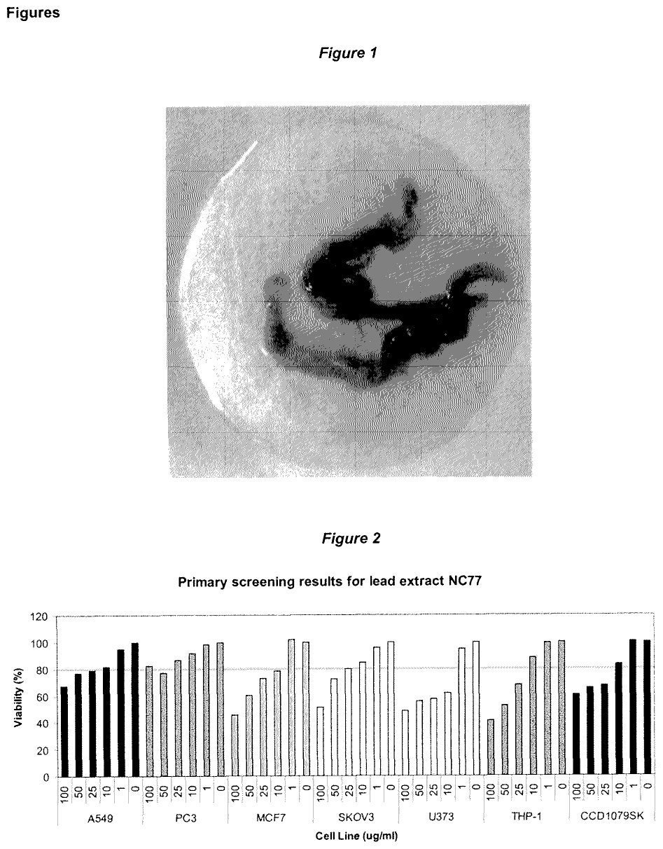

Prepared extracts were assessed following chronic exposure conditions in which cells were seeded at 2×10³ cells/well (96 wells-plate) and incubated with test compounds for 72 h. Each compound was evaluated over a range of concentrations (0, 10, 25, 50 or 100 μg/ml). Cell proliferation was initially assessed using a standard colorimetric indicator of metabolic activity (CIMA) assay. In this assay, tetrazole reduction was evaluated as a measure of metabolic function that evaluates mitochondrial activity to determine the extent of cell proliferation within a culture. This assay is based on the reduction of yellow tetrazolium salt to purple formazan by mitochondrial reductases enzyme in viable cells, resulting in a color change that confers a change in absorbance. Seven human cell lines were selected for primary evaluation: U373 (glioblastoma-astrocytoma), A549 (lung carcinoma), THP-1 (acute monocytic leukemia), MCF7 (mammary gland adenocarcinoma), SKOV3 (ovarian adenocarcinoma), PC3 (prostate adenocarcinoma) and CCD1079SK (fibroblast, noncancerous but proliferating) (see FIG. 2 and Table 2). Results are expressed in Table 2 as % viability (means±standard deviation of three replicates), with LD₅₀/IC₅₀ values in Tables 3 and 6. The most active results are expressed in bold. The maximum DMSO concentration was 1% at 100 μg/ml with 5% SDS used as a known toxic inducer.

From the results presented in Table 2, extract #1 (NC77) was selected for additional fractionation and evaluated in the same manner.

TABLE 2
In vitro screening of extracts #1 (NC77) and #2 (NC107).

| | |
|---|---|
| Extract #1 | Extract #2 |
| NC 77 | NC107 |

| Cell line (ug/ml) | | AVG | SD | AVG | SD |
|---|---|---|---|---|---|
| A549 | 100 | 67.5 | 1.6 | 70.5 | 2.5 |
| | 50 | 77.1 | 2.4 | 88.8 | 3.2 |
| | 25 | 78.8 | 3.1 | 91.3 | 3.6 |
| | 10 | 81.7 | 2.7 | 93.2 | 3.6 |
| | 1 | 94.6 | 4.6 | 102.5 | 6.8 |
| | 0 | 100.0 | 0.0 | 100.0 | 0.0 |
| | DMSO 1% | 92.5 | 3.1 | 92.5 | 3.1 |
| | SDS 5% | 68.7 | 6.6 | 68.7 | 6.6 |
| PC3 | 100 | 82.5 | 2.2 | 81.1 | 3.0 |
| | 50 | 77.2 | 4.0 | 87.1 | 2.3 |
| | 25 | 86.4 | 3.4 | 90.3 | 2.8 |
| | 10 | 91.7 | 2.0 | 94.9 | 0.8 |
| | 1 | 98.2 | 1.3 | 97.9 | 5.0 |
| | 0 | 100.0 | 0.0 | 100.0 | 0.0 |
| | DMSO 1% | 87.0 | 4.0 | 87.0 | 4.0 |
| | SDS 5% | 77.3 | 13.3 | 77.3 | 13.3 |
| MCF7 | 100 | 45.6 | 1.0 | 48.7 | 4.5 |
| | 50 | 60.3 | 2.6 | 71.2 | 4.9 |
| | 25 | 72.7 | 3.6 | 88.5 | 8.4 |
| | 10 | 78.7 | 3.9 | 94.0 | 8.5 |
| | 1 | 101.8 | 12.8 | 102.7 | 3.8 |
| | 0 | 100.0 | 0.0 | 100.0 | 0.0 |
| | DMSO 1% | 94.7 | 6.2 | 94.7 | 6.2 |
| | SDS 5% | 44.1 | 8.0 | 44.1 | 8.0 |
| SKOV3 | 100 | 50.9 | 4.6 | 45.1 | 5.6 |
| | 50 | 72.3 | 3.7 | 67.8 | 7.4 |
| | 25 | 79.8 | 4.8 | 76.4 | 5.9 |
| | 10 | 84.7 | 4.0 | 79.5 | 6.2 |
| | 1 | 95.5 | 5.7 | 93.6 | 9.5 |
| | 0 | 100.0 | 0.0 | 100.0 | 0.0 |
| | DMSO 1% | 95.3 | 7.5 | 95.3 | 7.5 |
| | SDS 5% | 78.9 | 3.9 | 20.0 | 78.9 |
| U373 | 100 | 48.4 | 2.0 | 34.5 | 3.1 |
| | 50 | 55.7 | 3.0 | 54.4 | 2.4 |
| | 25 | 57.0 | 2.8 | 61.3 | 4.4 |
| | 10 | 61.8 | 4.6 | 76.2 | 4.6 |
| | 1 | 94.6 | 2.7 | 96.6 | 6.9 |
| | 0 | 100.0 | 0.0 | 100.0 | 0.0 |
| | DMSO 1% | 83.4 | 7.7 | 83.4 | 7.7 |
| | SDS 5% | 48.4 | 6.4 | 48.4 | 6.4 |
| THP-1 | 100 | 40.8 | 1.2 | 35.8 | 1.4 |
| | 50 | 52.2 | 1.7 | 47.7 | 1.8 |
| | 25 | 67.9 | 4.4 | 56.6 | 4.8 |
| | 10 | 88.2 | 3.7 | 82.4 | 9.4 |
| | 1 | 99.4 | 7.2 | 105.6 | 5.8 |
| | 0 | 100.0 | 0.0 | 100.0 | 0.0 |
| | DMSO 1% | 89.2 | 3.6 | 89.2 | 3.6 |
| | SDS 5% | 20.3 | 4.0 | 20.3 | 4.0 |
| CCD1079SK | 100 | 60.3 | 4.6 | 46.1 | 1.1 |
| | 50 | 65.5 | 4.5 | 55.7 | 1.4 |
| | 25 | 67.3 | 3.6 | 62.7 | 2.7 |
| | 10 | 82.8 | 7.6 | 76.5 | 4.0 |
| | 1 | 100.3 | 10.3 | 92.8 | 3.1 |
| | 0 | 100.0 | 0.0 | 100.0 | 0.0 |
| | DMSO 1% | 91.3 | 7.8 | 91.3 | 7.8 |
| | SDS 5% | 9.4 | 0.9 | 9.4 | 0.9 |

TABLE 3
IC₅₀ calculations for crude extracts NC77 and NC 107 (μg/ml)

| | NC77 | NC107 |
|---|---|---|
| A549 | — | — |
| PC3 | — | — |
| MCF7 | 81.9 | 95.9 |
| SKOV3 | — | 88.2 |
| U373 | 66.1 | 50.8 |
| THP1 | 75.9 | 45.1 |
| CCD1079SK | — | 86.6 |

— LD50 > 1-μg/ml

### 3.4 Fractionation

Crude extract #1 was fractionated into five fractions by C-18 SPE column separation using 15 ml each of 5% methanol (fraction 1), 25% methanol (fraction 2), 50% methanol (fraction 3), 100% methanol (fraction 4) and methanol:dichloromethane (1:1) (fraction 5). Extracts were fractionated and the resulting fractions were evaluated using CIMA assay. Bio-assay guided fractionation provided detailed information regarding the compound(s) responsible for specific bioactivity.

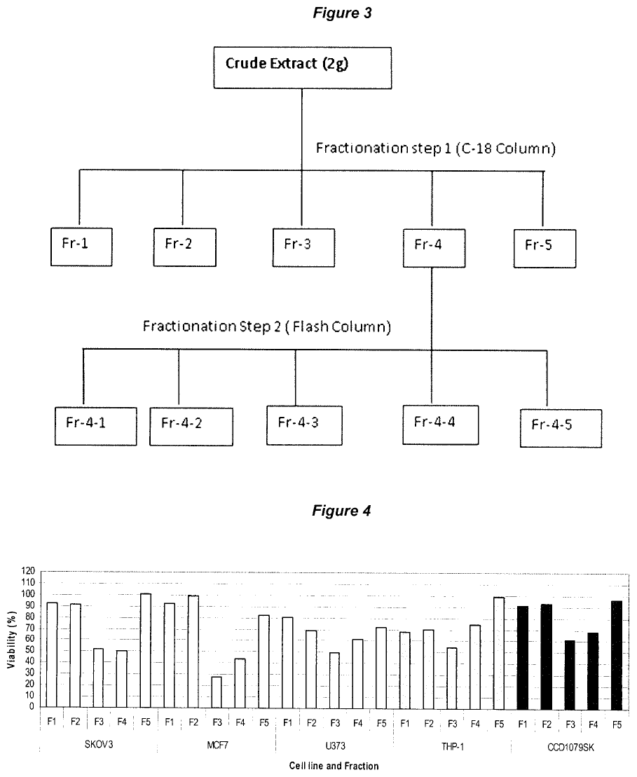

The following amounts were obtained from the strategy described in FIG. 3 and are summarized in Table 4.

TABLE 4
Amounts obtained for each fraction of NC77.
Sample (g)

| Extract 1 | 2.04 |
|---|---|
| F1 | 1.439 |
| F2 | 0.156 |
| F3 | 0.076 |
| F4 | 0.212 |
| F5 | 0.191 |

### 3.4.1 Fraction Results

The resulting fractions were evaluated using CIMA assay. Bio-assay guided fractionation provided detailed information regarding the compound(s) responsible for specific bioactivity (Table 5). Table 6 indicates the IC₅₀ of each fraction.

Also shown in FIG. 4, activity was in fractions 3 and 4. Considering the yields obtained for each fraction, the fractionation results clearly indicated that anti-cancer activity was distributed in F4 and priority was established for the selection for extract #1 (fraction 4). Fraction 4 (0.34 g) from NC77 was thus subjected to further sub-fractionation as presented below.

TABLE 5
Identification of active fractions from extract #1

| | | NC 77 | | | | | | | | | |
|---|---|---|---|---|---|---|---|---|---|---|---|
| | | F1 | | F2 | | F3 | | F4 | | F5 | |
| Cell line (ug/ml) | | AVG | SD | AVG | SD | AVG | SD | AVG | SD | AVG | SD |
| SKOV3 | 100 | 92.4 | 8.9 | 91.4 | 2.3 | 51.3 | 7.0 | 50.1* | 5.8 | 100.8 | 10.8 |
| | 50 | 104.2 | 12.5 | 92.6 | 3.5 | 77.0 | 11.0 | 53.8# | 3.4 | 96.3 | 4.3 |
| | 25 | 100.0 | 6.3 | 94.0 | 0.6 | 87.5 | 9.7 | 60.7* | 6.2 | 95.1 | 12.1 |
| | 10 | 101.0 | 11.1 | 99.5 | 3.9 | 91.4 | 10.9 | 68.3* | 4.7 | 93.9 | 12.9 |
| | 1 | 103.6 | 8.2 | 102.3 | 5.3 | 100.9 | 12.3 | 95.0 | 2.5 | 108.4 | 9.9 |
| | 0 | 100.0 | 0.0 | 100.0 | 0.0 | 100.0 | 0.0 | 100.0 | 0.0 | 100.0 | 0.0 |
| | DMSO 1% | 88.6 | 3.7 | 88.6 | 3.7 | 88.6 | 3.7 | 88.6 | 3.7 | 88.6 | 3.7 |
| | SDS 5% | 56.5 | 5.4 | 56.5 | 5.4 | 56.5 | 5.4 | 56.5 | 5.4 | 56.5 | 5.4 |
| MCF7 | 100 | 92.6 | 11.2 | 98.8 | 4.4 | 27.8 | 2.5 | 43.2# | 5.4 | 82.3 | 2.5 |
| | 50 | 98.4 | 4.9 | 99.4 | 7.3 | 52.5 | 6.2 | 50.3* | 3.0 | 91.1 | 2.9 |
| | 25 | 105.8 | 6.4 | 100.0 | 2.8 | 92.9 | 8.2 | 73.3* | 7.9 | 96.7 | 7.5 |
| | 10 | 103.4 | 2.2 | 106.8 | 5.4 | 101.1 | 4.2 | 80.4 | 15.2 | 96.0 | 3.7 |
| | 1 | 103.3 | 6.4 | 101.1 | 3.6 | 105.7 | 4.7 | 97.1 | 11.3 | 98.9 | 2.9 |
| | 0 | 100.0 | 0.0 | 100.0 | 0.0 | 100.0 | 0.0 | 100.0 | 0.0 | 100.0 | 0.0 |
| | DMSO 1% | 92.1 | 5.9 | 92.1 | 5.9 | 92.1 | 5.9 | 92.1 | 5.9 | 92.1 | 5.9 |
| | SDS 5% | 49.9 | 5.6 | 49.9 | 5.6 | 49.9 | 5.6 | 49.9 | 5.6 | 49.9 | 5.6 |
| U373 | 100 | 81.1 | 3.6 | 69.0 | 6.3 | 49.2 | 3.1 | 60.7 | 4.2 | 72.2 | 1.9 |
| | 50 | 85.3 | 8.4 | 82.3 | 5.9 | 83.0 | 5.8 | 87.2 | 5.4 | 84.4 | 3.1 |
| | 25 | 85.4 | 9.2 | 92.1 | 7.9 | 102.6 | 9.2 | 93.4 | 6.9 | 90.3 | 2.0 |
| | 10 | 103.9 | 6.3 | 103.2 | 10.1 | 102.2 | 8.1 | 93.6 | 7.0 | 106.0 | 2.6 |
| | 1 | 103.1 | 12.3 | 101.6 | 8.2 | 97.8 | 6.2 | 103.2 | 14.9 | 117.7 | 7.4 |
| | 0 | 100.0 | 0.0 | 100.0 | 0.0 | 100.0 | 0.0 | 100.0 | 0.0 | 100.0 | 0.0 |
| | DMSO 1% | 94.7 | 6.2 | 94.7 | 6.2 | 94.7 | 6.2 | 94.7 | 6.2 | 94.7 | 6.2 |
| | SDS 5% | 44.1 | 8.0 | 44.1 | 8.0 | 44.1 | 8.0 | 44.1 | 8.0 | 44.1 | 8.0 |
| THP-1 | 100 | 68.1 | 0.9 | 70.8 | 4.1 | 54.3 | 1.7 | 74.9 | 3.7 | 98.8 | 9.5 |
| | 50 | 79.5 | 3.2 | 78.2 | 8.4 | 75.5 | 4.1 | 78.8 | 4.1 | 99.1 | 10.9 |
| | 25 | 74.5 | 3.1 | 80.0 | 4.9 | 75.6 | 5.0 | 79.1 | 5.8 | 92.4 | 8.6 |
| | 10 | 75.9 | 3.7 | 78.9 | 5.1 | 75.0 | 3.1 | 81.3 | 3.2 | 93.2 | 7.5 |
| | 1 | 75.7 | 1.8 | 80.4 | 3.5 | 82.7 | 5.9 | 87.0 | 7.8 | 93.9 | 5.6 |
| | 0 | 100.0 | 0.0 | 100.0 | 0.0 | 100.0 | 0.0 | 100.0 | 0.0 | 100.0 | 0.0 |
| | DMSO 1% | 103.3 | 6.2 | 103.3 | 6.2 | 103.3 | 6.2 | 103.3 | 6.2 | 103.3 | 6.2 |
| | SDS 5% | 36.2 | 4.6 | 36.2 | 4.6 | 36.2 | 4.6 | 36.2 | 4.6 | 36.2 | 4.6 |
| CCD1079SK | 100 | 91.9 | 4.5 | 93.3 | 8.8 | 60.6 | 8.4 | 68.1 | 6.7 | 96.6 | 4.1 |
| | 50 | 102.4 | 8.2 | 103.0 | 12.1 | 85.6 | 8.6 | 76.8 | 6.0 | 100.1 | 4.5 |
| | 25 | 99.3 | 1.5 | 101.8 | 11.5 | 85.8 | 13.8 | 81.2 | 3.4 | 98.8 | 6.2 |
| | 10 | 100.7 | 5.1 | 99.5 | 13.5 | 93.8 | 8.1 | 84.0 | 1.5 | 96.9 | 5.1 |
| | 1 | 102.7 | 4.4 | 98.3 | 9.8 | 97.3 | 10.9 | 89.2 | 4.7 | 98.5 | 1.9 |
| | 0 | 100.0 | 0.0 | 100.0 | 0.0 | 100.0 | 0.0 | 100.0 | 0.0 | 100.0 | 0.0 |
| | DMSO 1% | 87.2 | 9.7 | 87.2 | 9.7 | 87.2 | 9.7 | 87.2 | 9.7 | 87.2 | 9.7 |
| | SDS 5% | 11.3 | 0.6 | 11.3 | 0.6 | 11.3 | 0.6 | 11.3 | 0.6 | 11.3 | 0.6 |

TABLE 6
IC₅₀ calculations for NC77 fractions (μg/ml)

| | NC77 | | | | |
|---|---|---|---|---|---|
| | F1 | F2 | F3 | F4 | F5 |
| SKOV3 | — | — | — | 80.2 | — |
| MCF7 | — | — | 67.9 | 73.9 | — |
| U373 | — | — | — | — | — |
| THP-1 | — | — | — | — | — |
| CCD1079SK | — | — | — | — | — |

—, LD50 > 100 μg/ml

### Example 4. Sub-Fractionation of Fraction 4 from Extract #1 (NC77)

Fraction 4 was selected for further sub-fractionation based on the bioassay results (Table 5). Thus, in a second stage, NC77-F4 was dissolved in dichloromethane/methanol and mixed with Celite and dried via rotary evaporation. The sample was loaded on 24 g Teledyne ISCO High Performance GOLD silica gel column and eluted with dichloromethane/methanol on CombiFlash® Rf, Teledyne ISCO. Sub-fractionation was conducted as a gradient of Solvent A (CH₂Cl₂) and Solvent B (1:1 methanol:water) as follows: 0% B for 2 CV (column volume) then to 40% B for 17 CV, to 100% B for 4CV for a total elution in 23 CV.

Fractions were monitored by TLC and some were combined and dried using Rotavap and Genevac to yield five (5) sub-fractions as presented in Table 7.

TABLE 7
Yields of sub-fractionation of NC77-F4

| | Sample weight | Fr. 1 | Fr. 2 | Fr. 3 | Fr. 4 | Fr. 5 |
|---|---|---|---|---|---|---|
| Sample | (g) | (g) | (g) | (g) | (g) | (g) |
| NC77 - F4 | 0.21 | 0.011 | 0.044 | 0.006 | 0.036 | 0.064 |

These sub-fractions were again subjected to the bioassay evaluation presented above. Results are expressed in Table 8 as % viability (means±standard deviation of three replicates), with LD₅₀/10₅₀ values in Table 9. The most active results are expressed in bold with statistical analyses indicated. The maximum DMSO concentration was 1% at 100 μg/ml with 5% SDS used as a known toxic inducer.

Results clearly indicated that anti-cancer activity was distributed in sub-fractions F3 and F4 from fraction 4 of NC77.

TABLE 8
In vitro screening results - sub-fractions from NC77-F4

| | | NC77 | | | | | | | | | |
|---|---|---|---|---|---|---|---|---|---|---|---|
| | | F1 | | F2 | | F3 | | F4 | | F5 | |
| Cell line (ug/ml) | | AVG | SD | AVG | SD | AVG | SD | AVG | SD | AVG | SD |
| PC3 | 100 | 104.4 | 3.4 | 92.4 | 4.2 | 89.7 | 6.8 | 98.9 | 6.0 | 87.9 | 2.6 |
| | 50 | 116.2 | 21.2 | 93.3 | 7.3 | 93.3 | 8.3 | 93.2 | 6.1 | 89.3 | 3.9 |
| | 25 | 120.9 | 13.8 | 97.6 | 8.6 | 97.9 | 9.0 | 98.4 | 1.9 | 95.0 | 4.5 |
| | 10 | 115.4 | 9.5 | 99.6 | 8.5 | 99.3 | 6.1 | 101.4 | 3.5 | 96.1 | 6.0 |
| | 1 | 120.9 | 14.2 | 96.7 | 10.9 | 101.7 | 9.9 | 101.6 | 8.0 | 105.5 | 3.4 |
| | 0 | 100.0 | 0.0 | 100.0 | 0.0 | 100.0 | 0.0 | 100.0 | 0.0 | 100.0 | 0.0 |
| | DMSO 1% | 7.7 | 0.8 | 7.7 | 0.8 | 7.7 | 0.8 | 7.7 | 0.8 | 7.7 | 0.8 |
| | SDS 5% | 94.4 | 6.1 | 94.4 | 6.1 | 94.4 | 6.1 | 94.4 | 6.1 | 94.4 | 6.1 |
| SKOV3 | 100 | 91.0 | 8.2 | 91.2 | 4.4 | 86.6 | 1.9 | 92.0 | 8.0 | 84.6 | 2.6 |
| | 50 | 97.7 | 0.8 | 102.8 | 2.2 | 95.3 | 2.1 | 101.7 | 3.9 | 86.7 | 2.6 |
| | 25 | 100.0 | 1.3 | 103.3 | 1.7 | 100.0 | 4.5 | 103.2 | 2.0 | 86.1 | 4.5 |
| | 10 | 99.0 | 3.8 | 102.2 | 0.7 | 99.8 | 0.9 | 101.2 | 4.0 | 91.0 | 1.6 |
| | 1 | 98.1 | 1.3 | 103.6 | 2.6 | 100.5 | 6.5 | 99.7 | 1.0 | 97.0 | 3.6 |
| | 0 | 100.0 | 0.0 | 100.0 | 0.0 | 100.0 | 0.0 | 100.0 | 0.0 | 100.0 | 0.0 |
| | DMSO 1% | 14.3 | 0.7 | 14.3 | 0.7 | 14.3 | 0.7 | 14.3 | 0.7 | 14.3 | 0.7 |
| | SDS 5% | 90.7 | 33.4 | 90.7 | 33.4 | 90.7 | 33.4 | 90.7 | 33.4 | 90.7 | 33.4 |
| A549 | 100 | 100.1 | 11.7 | 109.9 | 12.7 | 88.7 | 12.2 | 98.2 | 11.1 | 88.2 | 5.4 |
| | 50 | 107.9 | 3.7 | 103.3 | 10.5 | 99.7 | 10.7 | 110.6 | 2.3 | 83.0 | 4.6 |
| | 25 | 110.8 | 5.4 | 105.9 | 5.1 | 104.6 | 9.5 | 117.5 | 3.7 | 81.6 | 7.1 |
| | 10 | 112.9 | 2.1 | 109.5 | 5.0 | 106.3 | 9.6 | 117.1 | 12.1 | 94.5 | 11.9 |
| | 1 | 106.1 | 4.0 | 112.8 | 12.5 | 105.0 | 6.0 | 117.4 | 5.6 | 94.7 | 8.7 |
| | 0 | 100.0 | 0.0 | 100.0 | 0.0 | 100.0 | 0.0 | 100.0 | 0.0 | 100.0 | 0.0 |
| | DMSO 1% | 6.4 | 0.6 | 6.4 | 0.6 | 6.4 | 0.6 | 6.4 | 0.6 | 6.4 | 0.6 |
| | SDS 5% | 66.2 | 13.3 | 66.2 | 13.3 | 66.2 | 13.3 | 66.2 | 13.3 | 66.2 | 13.3 |
| THP-1 | 100 | 11.0 | 0.5 | 12.5 | 1.2 | 15.0 | 1.6 | 27.2 | 4.0 | 19.1 | 2.0 |
| | 50 | 37.5 | 1.9 | 39.3 | 1.1 | 26.9 | 3.6 | 57.1 | 4.7 | 59.8 | 8.6 |
| | 25 | 61.9 | 1.4 | 71.8 | 5.9 | 66.5 | 8.3 | 67.5 | 2.2 | 85.5 | 5.5 |
| | 10 | 99.1 | 5.1 | 78.0 | 8.9 | 72.9 | 9.2 | 76.3 | 4.5 | 87.9 | 6.1 |
| | 1 | 102.2 | 10.6 | 96.8 | 8.6 | 104.3 | 12.7 | 78.2 | 7.2 | 96.5 | 5.0 |
| | 0 | 100.0 | 0.0 | 100.0 | 0.0 | 100.0 | 0.0 | 100.0 | 0.0 | 100.0 | 0.0 |
| | DMSO 1% | 1.0 | 0.5 | 1.0 | 0.5 | 1.0 | 0.5 | 1.0 | 0.5 | 1.0 | 0.5 |
| | SDS 5% | 39.9 | 2.9 | 39.9 | 2.9 | 39.9 | 2.9 | 39.9 | 2.9 | 39.9 | 2.9 |
| U373 | 100 | 6.0 | 1.4 | 3.3 | 0.8 | 1.4 | 0.5 | 28.1 | 3.5 | 5.3 | 0.7 |
| | 50 | 79.6 | 8.4 | 109.0 | 14.2 | 43.4 | 7.0 | 67.4 | 4.5 | 61.5 | 9.4 |
| | 25 | 92.5 | 30.1 | 113.0 | 2.9 | 88.2 | 5.5 | 72.1 | 2.0 | 66.0 | 8.5 |
| | 10 | 108.4 | 18.5 | 102.6 | 9.6 | 103.5 | 13.8 | 76.1 | 4.1 | 88.7 | 8.2 |
| | 1 | 106.1 | 14.8 | 117.6 | 5.4 | 110.5 | 7.3 | 95.9 | 3.0 | 81.0 | 9.5 |
| | 0 | 100.0 | 0.0 | 100.0 | 0.0 | 100.0 | 0.0 | 100.0 | 0.0 | 100.0 | 0.0 |
| | DMSO 1% | 3.0 | 0.4 | 3.0 | 0.4 | 3.0 | 0.4 | 3.0 | 0.4 | 3.0 | 0.4 |
| | SDS 5% | 78.5 | 11.6 | 78.5 | 11.6 | 78.5 | 11.6 | 78.5 | 11.6 | 78.5 | 11.6 |
| CCD1079SK | 100 | 22.5 | 3.1 | 8.2 | 0.9 | 9.4 | 1.9 | 32.9 | 1.8 | 66.6 | 13.3 |
| | 50 | 54.2 | 0.5 | 89.4 | 6.7 | 9.3 | 3.5 | 60.3 | 4.6 | 82.6 | 6.1 |
| | 25 | 89.8 | 1.8 | 95.5 | 2.7 | 58.1 | 5.2 | 81.1 | 8.6 | 76.3 | 7.5 |
| | 10 | 94.0 | 4.7 | 98.1 | 11.2 | 85.2 | 8.9 | 86.2 | 4.7 | 98.7 | 11.6 |
| | 1 | 101.5 | 4.8 | 95.6 | 0.9 | 101.7 | 8.7 | 98.8 | 6.9 | 96.3 | 4.1 |
| | 0 | 100.0 | 0.0 | 100.0 | 0.0 | 100.0 | 0.0 | 100.0 | 0.0 | 100.0 | 0.0 |
| | DMSO 1% | 8.6 | 0.9 | 8.6 | 0.9 | 8.6 | 0.9 | 8.6 | 0.9 | 8.6 | 0.9 |
| | SDS 5% | 87.9 | 7.2 | 87.9 | 7.2 | 87.9 | 7.2 | 87.9 | 7.2 | 87.9 | 7.2 |
| MCF7 | 100 | 23.1 | 2.9 | 24.1 | 0.5 | 26.7 | 2.0 | 25.1 | 1.7 | 57.5 | 7.3 |
| | 50 | 42.3 | 3.7 | 69.3 | 3.2 | 56.4 | 8.1 | 45.2 | 4.6 | 103.6 | 12.1 |
| | 25 | 83.7 | 9.0 | 92.5 | 2.3 | 84.3 | 8.4 | 81.8 | 5.1 | 108.6 | 4.0 |
| | 10 | 93.6 | 7.2 | 91.4 | 1.2 | 101.0 | 11.8 | 84.3 | 0.5 | 104.3 | 6.8 |
| | 1 | 96.7 | 2.6 | 98.6 | 6.6 | 98.7 | 9.0 | 87.3 | 3.3 | 107.3 | 4.7 |
| | 0 | 100.0 | 0.0 | 100.0 | 0.0 | 100.0 | 0.0 | 100.0 | 0.0 | 100.0 | 0.0 |
| | DMSO 1% | 16.9 | 3.3 | 16.9 | 3.3 | 16.9 | 3.3 | 16.9 | 3.3 | 16.9 | 3.3 |
| | SDS 5% | 93.7 | 8.7 | 93.7 | 8.7 | 93.7 | 8.7 | 93.7 | 8.7 | 93.7 | 8.7 |

TABLE 9
LD₅₀ calculations sub-fractions from NC77-F4 (μg/ml)

| Cell line (ug/ml) | F1 | F2 | F3 | F4 | F5 |
|---|---|---|---|---|---|
| PC3 | — | — | — | — | — |
| SKOV3 | — | — | — | — | — |
| A549 | — | — | — | — | — |
| THP-1 | 31.1 | 48.8 | 34.0 | 58.8 | 62.3 |
| U373 | 68.6 | — | 49.1 | 66.9 | 50.4 |
| CCD1079SK | 64.3 | — | 22.2 | 71.1 | — |
| MCF7 | 59.4 | 70.4 | 58.1 | 50.6 | — |

### Example 5. Characterization of a Main Component from NC77 and NC77-F4

### 5.1. Sample Preparation and Analysis:

### FAME Preparation and GC-MS Analysis

Extract #1 (NC77) and its fraction-4 (NC77-Fr. 4) were analyzed by GC-MS. Samples were added 1 mL sodium methoxide solution and 1 mL hexane, with cap closed. The reaction vials were put in a heating block (80° C.) for 15 min shaking vials with hand at 5 min intervals. Added 1 mL saturated NaCl solution after cooling to room temperature and shaken by hand several times. The reaction vials were then centrifuged for 20 min at 2,000 rpm. The upper solutions were transferred to GC vials. For GC-MS analysis, Agilent 6890 with 5973 Mass Selective Detector were used. The column was Agilent DB-23 (59 m×0.25 mm, 0.15 μm), injection volume 1 μL. Oven program: 50° C. for 1 min, 25° C./min to 170° C., 2.75° C./min to 215° C. (hold 12 min), 40° C./min to 230° C. (hold 3.11 min). Total runtime was 37.65 min. FID temperature was 280° C., hydrogen flow 40 mL/min, air flow 400 mL/min, makeup flow N2 20 mL/min. Split ratio was 2:1. Carrier gas was helium and kept at constant pressure (30 psi). MSD ionization mode was El. Interface temperature 250° C., MS source 230° C., MS Quad 150° C. Mass range was 50-600 m/z.

### HPLC-DAD Analysis to Compare Different Batches

The separation was conducted on an Agilent Zorbax SB-C18 (2.1×30 mm 3.5 μm) column using Agilent HPLC 1100. Solvent A was 10 mM ammonium formate (pH 3.2) and solvent B was 90% acetonitrile with 10% 100 mM ammonium formate (pH3.2). Gradient was 30% B to 100% B in 12 min and wash with acetonitrile with 0.1% formic acid for 2 min. Column temperature was 55° C. Flow rate was 0.5 mL/min.

### HPLC-DAD/MS Analysis of Carotenoids

Analysis was done on Agilent 1200 system using YMC carotenoid column, 0. 5 μm (250×2 mm) at 32° C. Mobile phase: solvent A 50 mM AmAc/MeOH, Solvent B MTBE, with gradient of 5-65% B in 40 min. Flow rate was 0.2 mL/min, and DAD detector monitored at 450 nm. Identification of peaks in the chromatogram was made on the basis of RT comparison to known standards: fucoxanthin, astaxanthin, lutein, zeaxanthin, canthaxanthin, α-& β-Carotene. All standards were purchased from Chromadex.

### UPLC-DAD/ELSD/HRMS and MS/MS

The following instruments were used for LC-UV-ELSD-HRMS data acquisition: Accela 1250 pump (Thermo Fisher Scientific); Exactive benchtop Orbitrap mass spectrometer (Thermo Fisher) equipped with heated electrospray ionization probe; Utimate 3000 DAD (Thermo Scientific Dionex) and ELSD 3300 (Alltech). Separation was carried out on a Hypersil C18 column (50×2.1 mm, Thermo) using mobile phase consisted of (A) 0.1% formic acid and (B) 0.1% formic acid in acetonitrile, with a linear gradient from 5% B to 100% B in 4.2 min, held for 3.2 min, flow-rate was at 400 μL/min.

HRMS was acquired in positive or negative polarity at 25,000 resolution, each with a HCD scan with collision energy at 50 eV for all-ion-fragmentations with 10,000 resolution. The following optimal ion source conditions were used: sheath flow is 15, auxiliary gas flow rate of 3; spray voltage of 3 kV (−2.5 kV for negative); capillary and heater temperature of 350° C. and 250° C., respectively.

### NMR

The samples were reconstituted in 100 μL of CDCl₃ and 60 μL of each sample was transferred to 1.7 mm NMR tubes. All spectra were run on a Bruker Avance III 700 MHz spectrometer equipped with a 1.7 mm cryogenically cooled probe operating at 16K.

### 5.2. Analysis of NC77 and NC77-Fr. 4

The preliminary profiling work done previously indicated that lipids were the main components in NC77-Fr. 4. As such, GC-MS analysis was performed to understand the fatty acids composition in this extract and its bioactive fraction.

FIG. 5 is the GC chromatogram of FAME prepared from extract #1 (NC77) and #1-F4 (NC77-Fr. 4). The retention time and tentative identification based on NIST database matching are presented in Table 10. The major fatty acid was shown to be palmitic acid.

TABLE 10
Identification of fatty acids using GC-MS analysis of extract
#1 (NC77) and extract #1-F4 (NC77-Fr. 4)

| RT (min) | Compounds |
|---|---|
| 11.32 | Methyl tetradecanoate |
| 13.51 | Hexadecanoic acid, methyl ester |
| 13.87 | 9-Hexadecenoir acid, methyl ester, (Z)- |
| 15.49 | unknown |
| 16.71 | 8-Octadecenoic acid, methyl ester |
| 16.83 | 8-Octadecenoic acid, methyl ester |
| 17.56 | 9,12-Octadecadienoic acid (Z, Z)-, methyl ester |
| 18.66 | 1,2-15,16-Diepoxyhexadecane |
| 23.47 | Methyl eicosa-5,8,11,14,17-pentaenoate (EPA) |

We also compared the NMR and HPLC profiles of two different batches of NC77 and NC77-Fr. 4 prepared and tested earlier, with new samples.

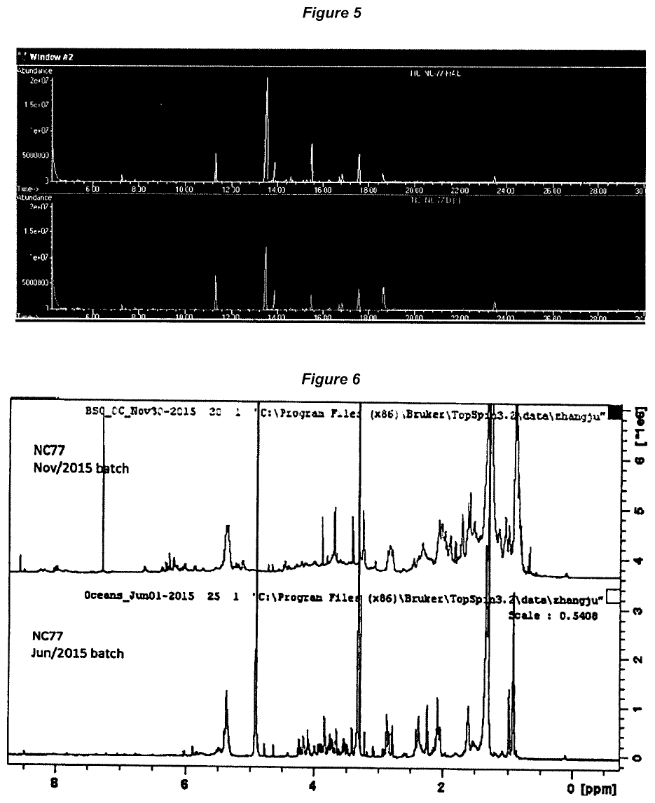

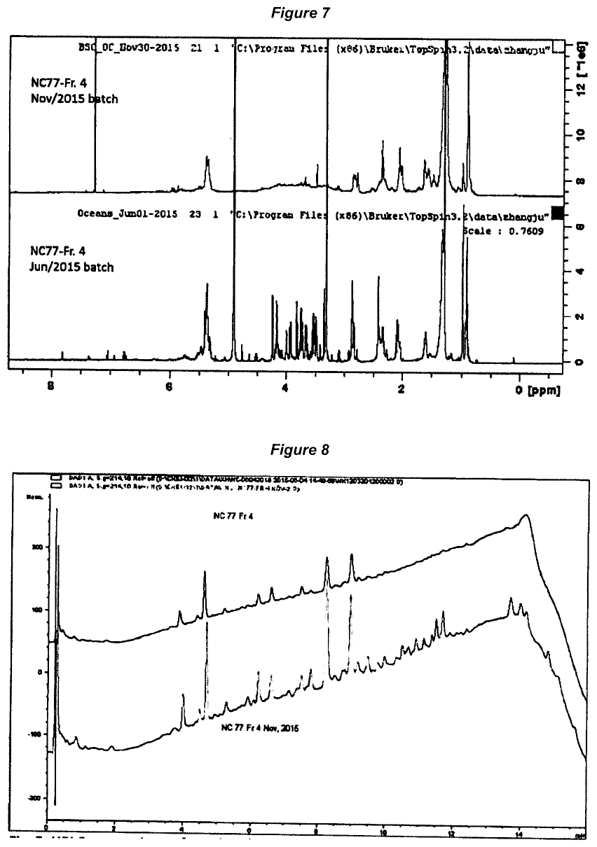

As shown in FIGS. 6 and 7, extract #1 and its fraction 4 (NC77 and NC77-Fr. 4) prepared in November 2015 have similar ¹H-NMR profiles as the ones observed from the earlier batch (June 2015). Lipids (fatty acid) appeared to be the major component.

HPLC comparison is shown in FIG. 8. The two appeared to have similar profiles of main components.

### Example 6. Fractionation and Purification of Main Components from NC77-F4-F13

In a second series of experiments, NC77 was fractionated as above and fraction 4 was sub-fractioned but with a slightly different protocol to obtain better compound separation. In short, 3.14 g of extract #1 (NC77) was dissolved in methanol, mixed on Celite and dried using Rotavap. The sample was then loaded on pre-conditioned and equilibrated Thermo Scientific SPE column (HYPERSEP C18 20G). Four fractions were obtained by eluting the SPE column with 5% methanol (Fr. 1), 25% methanol (Fr. 2), 50% methanol (Fr. 3) and 100% methanol (Fr. 4).

Based on previous bioassay result, fraction-4 (0.34 g) was subjected to further fractionation. Fraction 4 was dissolved in dichloromethane/methanol and mixed with Celite and dried. The sample was loaded on 24 g Teledyne ISCO High Performance GOLD silica gel column and eluted with dichloromethane/methanol on CombiFlash® Rf, Teledyne ISCO. The eluting solvent gradient (A and B) was as the following: 0% B for 2 CV (column volume) then to 40% B for 15 CV and kept at 40% B for 2 CV, to 100% B for 2 CV and kept at 100% B for 2 CV. Total elution volume was 23 CV. A is dichloromethane and B is methanol/dichloromethane (1:1). Fractions were monitored by TLC and some combined and dried using Rotavap and Genevac.

Based on TLC analysis, sub-fraction 13 of Fr. 4 (Fr. 4-13, 0.11 g) was chosen for subsequent purification as it showed to contain major components. In this step, 12 g silica gel column by CombiFlash® Rf was used. The solvent (A and B) gradient was: 0% B for 2 CV then to 100% B for 25 CV and kept at 100% B for 2 CV. The total elution was 29 CV. A is dichloromethane and B is 5% methanol in dichloromethane. Again, fractions were selectively combined according to TLC.

Three sub-fractions were further purified using semi preparative HPLC (Agilent). The column used was ZORBAX SB-C18 (9.4×50 mm, 5 μm) and the mobile phase was water/acetonitrile. Eluting gradient varied for different samples so to optimize separation. The column temperature was at 55° C. and flow rate 5 mL/min.

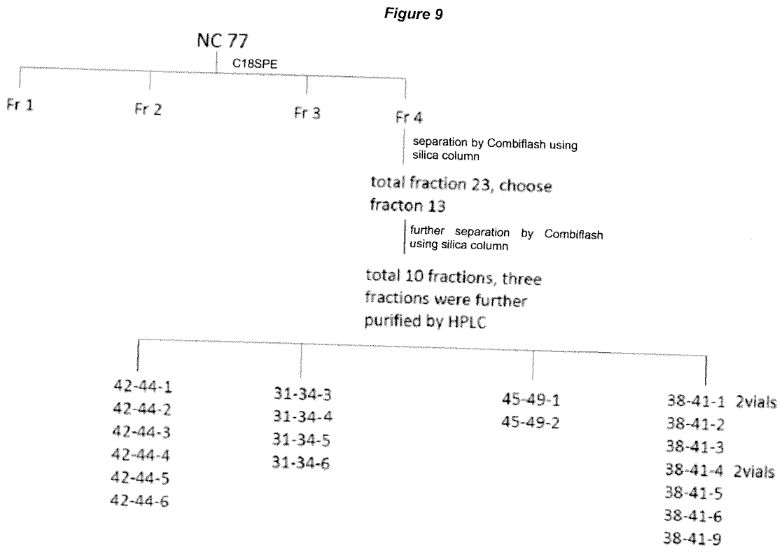

As shown in FIG. 9, the above described fractionation and purification yielded 19 samples for full structural analysis. Among them, sample (II) (3.9 mg) was a pure compound.

### Example 7. Structure of Pure Compound (I)

### 7.1. Compound (I)

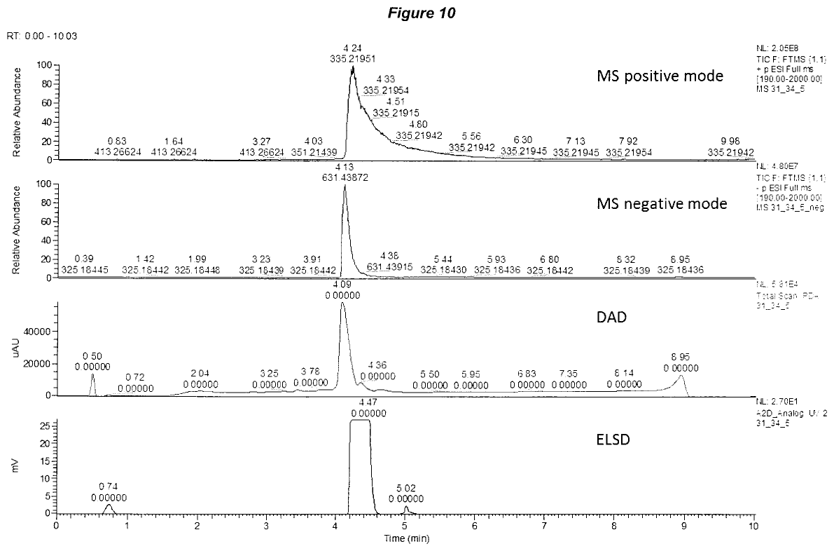

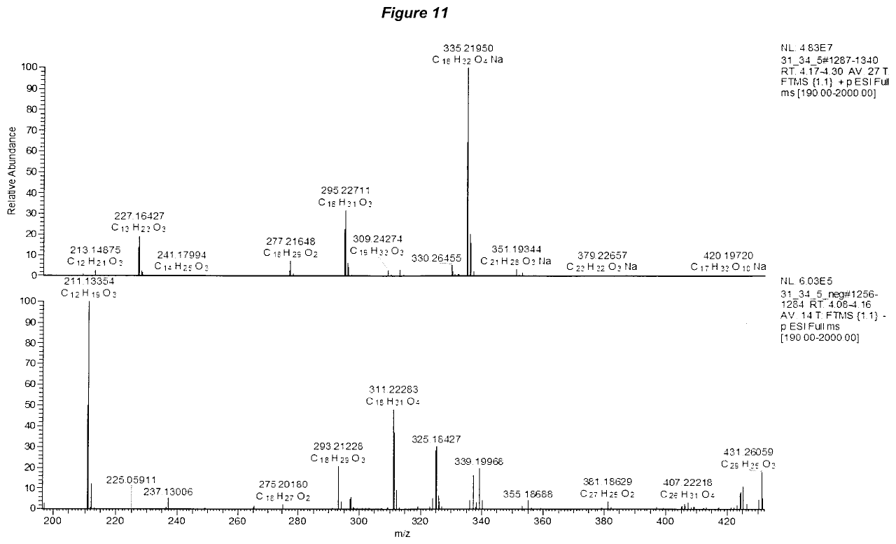

UPLC-DAD/ELSD/HRMS data (FIG. 10) revealed compound (I) as a pure compound. HRMS (FIG. 11) peaks of m/z 335.21950 (positive mode, observed for C₁₈H₃₂O₄Na⁺, calculated 335.21983) and m/z 311.22283 (negative mode, observed for C₁₈H₃₁O₄⁻, calculated 311.22223) confirmed the molecular formula of C₁₈H₃₂O₄.

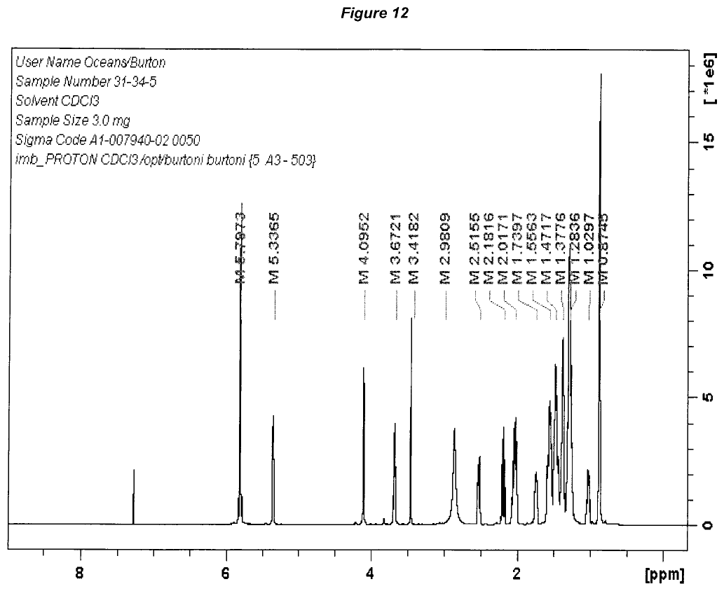

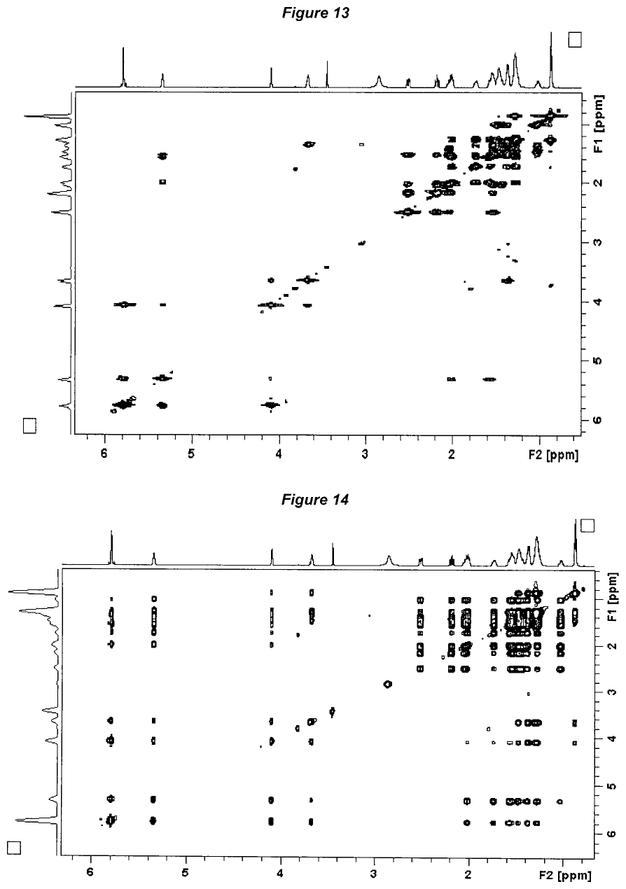

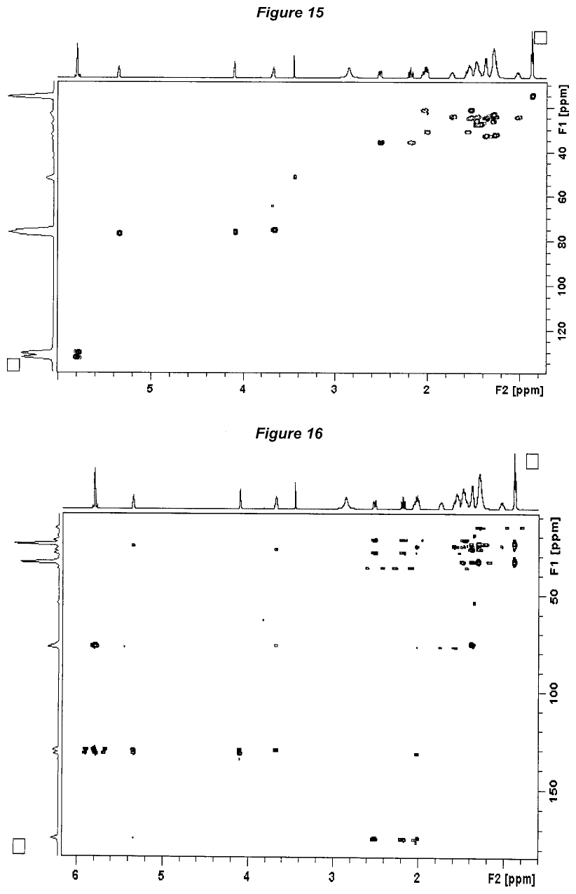

¹H-NMR, COSY, TOCSY, and HSQC spectra (FIGS. 12-15) showed the presence of 2 olefinic methines (55.79 ppm), 3 oxygenated methines (δ5.34, 4.09, 3.67 ppm), 11 methylenes (52.6-1.0 ppm), and 1 terminal methyl group (δ0.87 ppm).

HMBC (FIG. 16) revealed the key C—H correlations that lead to proposing 12,13-dihydroxy-10-octadecen-9-olide as the structure for compound (I) (arrows for key HMBC correlations):

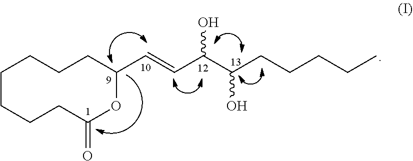 [SMILES: CCCCCC(O)C(O)/C=C/C1CCCCCCCC(=O)O1]

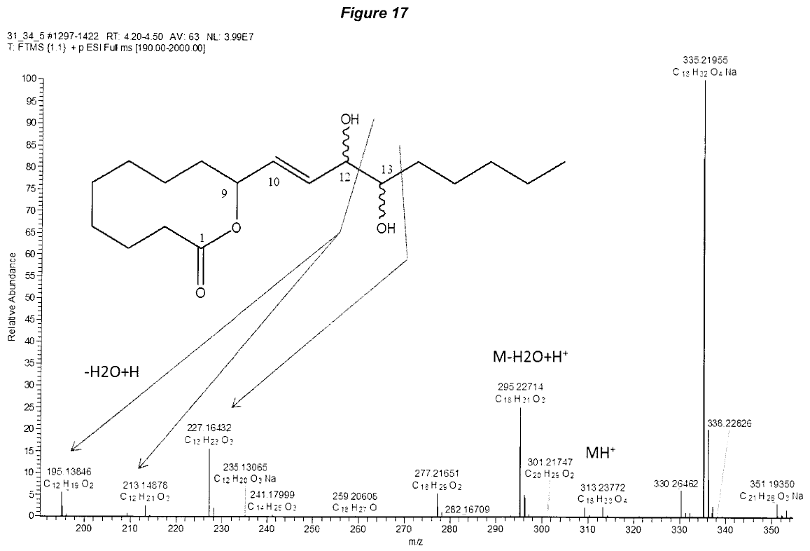

The double bond at C-10,11 was determined to be transformed based on the coupling J value of the olefinic protons. HRMS (FIG. 17) fragment ions also confirmed the structure.

Based on databases structure search, compound (I) is a novel compound. It is a fatty acid derivative, likely formed through esterification of unsaturated and oxygenated octadecanoic acid or stearic acid (18:0). Full spectral data assignment is listed in Table 11.

TABLE 11
NMR data of compound (I)

| | dC, | | dH, | |
|---|---|---|---|---|
| Position | ppm | type | ppm (J in Hz) | |
| 1 | 175.3 | C═O | | |
| 2 | 36.7 | CH₂ | 2.51 | ddd (15.9; 6.4; 3.3) |
| | | | 2.18 | td (13.6; 2.8) |
| 3 | 21.7 | CH₂ | 2.04 | m |
| | | | 1.54 | m |
| 4 | 28.2 | CH₂ | 1.45 | m |
| 5 | 25.3 | CH₂ | 1.54 | m |
| | | | 1.36 | m |
| 6 | 24.9 | CH₂ | 1.45 | m |
| | | | 1.02 | m |
| 7 | 24.7 | CH₂ | 1.70 | m |
| | | | 1.23 | m |
| 8 | 31.4 | CH₂ | 1.97 | m |
| | | | 1.55 | m |
| 9 | 76.7 | CH | 5.34 | m |
| 10 | 132.6 | CH | 5.78 | dd (15.9; 4.6) |
| 11 | 130.4 | CH | 5.76 | dd (15.9; 5.0) |
| 12 | 76.2 | CH | 4.08 | br · s |
| 13 | 75.3 | CH | 3.64 | br · s |
| 14 | 33.1 | CH₂ | 1.37 | m |
| 15 | 26.8 | CH₂ | 1.47 | m |
| | | | 1.29 | m |
| 16 | 32.9 | CH₂ | 1.29 | m |
| 17 | 23.6 | CH₂ | 1.29 | m |
| 18 | 14.7 | CH₂ | 0.87 | t (7.1) |

### Example 8. In Vitro Anti-Cancer Activity of Compound of Formula (I)

The in vitro anti-cancer activity of compound (I) is presented as average cell viability in Table 12, whereas Table 13 presents the same data but as fold change viability ±standard error.

TABLE 12
In vitro activity of compound (I) - average cell viability

| Sample # | Sample ID | Ug/mL | PC3 | A549 | U373 | SKOV | MDA-MB | CCD | THP-1 |
|---|---|---|---|---|---|---|---|---|---|
| 3 | (II) | 100 | 19 | 61 | 3 | 85 | 20 | 17 | 44 |
| | | 50 | 97 | 92 | 68 | 94 | 66 | 90 | 68 |
| | | 10 | 93 | 93 | 85 | 97 | 96 | 84 | 84 |
| | | 1 | 100 | 95 | 90 | 97 | 101 | 91 | 96 |

| | | | | | | | | |
|---|---|---|---|---|---|---|---|---|
| Vehicle cont-DMSO | 1% | 96 | 86 | 98 | 93 | 92 | 91 | 88 |
| Positive cont-SDS | 250 ug/ml | 8 | 11 | 4 | 9 | 14 | 5 | 16 |

TABLE 13
In vitro activity of compound (I) - fold change viability ± standard error

| | PC3 | | A549 | | U373 | | SKOV | |
|---|---|---|---|---|---|---|---|---|
| ug/mL | AVG | ERROR | AVG | ERROR | AVG | ERROR | AVG | ERROR |
| 100 | 0.19 | 0.02 | 0.72 | 0.11 | 0.03 | 0.00 | 0.92 | 0.01 |
| 50 | 1.01 | 0.06 | 1.08 | 0.02 | 0.69 | 0.06 | 1.01 | 0.05 |
| 10 | 0.96 | 0.01 | 1.09 | 0.01 | 0.86 | 0.02 | 1.05 | 0.02 |
| 1 | 1.03 | 0.12 | 1.11 | 0.07 | 0.92 | 0.04 | 1.04 | 0.01 |

| | MDA-MB | | CCD | | THP-1 | |
|---|---|---|---|---|---|---|
| ug/mL | AVG | ERROR | AVG | ERROR | AVG | ERROR |
| 100 | 0.21 | 0.01 | 0.18 | 0.02 | 0.50 | 0.06 |
| 50 | 0.72 | 0.08 | 0.99 | 0.09 | 0.77 | 0.05 |
| 10 | 1.05 | 0.06 | 0.92 | 0.02 | 0.95 | 0.01 |
| 1 | 1.10 | 0.05 | 1.00 | 0.03 | 1.09 | 0.03 |

(bold = decrease >25%).

### Example 9. Stereochemistry Determination of Compound (I)

### Materials and Methods

Compound (II) (i.e. NC77(2)-44-46 isolated from NC77) stereochemistry was determined using S-(+)-Methoxytrifluoromethylphenylacetyl chloride (S+MTPA-CI, 99.0%) and R-(−)-methoxytrifluoromethylphenylacetyl chloride (R-MTPA-CI, 99.0%) that were purchased from Sigma-Aldrich (Oakville, ON).

### Optical Rotation Measurement

Optical rotation of compound (II) was measured on a Perkin Elmer polarimeter model 341 (PerkinElmer Inc., UK). Both CHCl₃ and MeOH were used as solvents, at concentration 2.98 mg/mL and 1.79 mg/mL, respectively.

Preparation of Mosher's Ester from Compound (II)

The initial Mosher ester preparation was carried out using 2 mg NC77(2)-44-46, dissolved in 1 mL CHCl₃, added 0.5 mL pyridine and 60 μL of S+MTPA-CI or R-MTPA-Cl. The mixture was stirred in room temperature overnight, dried in N2. Mono-MTPA esters (S and R forms) were purified using analytical TLC (silica gel), yielding 0.5 mg of R-mono-MTPA ester (from S+MTPA-CI) and 0.6 mg of the S form (R-MTPA-CI), as well as remaining compound NC77(2)-44-46.

To repeat the preparation of Mosher esters, 4 mg of NC77(2)-44-46 was dissolved in 200 μL CHCl₃ (dried with molecular sieve), added 8 μL pyridine (dried with molecular sieve) and 60 μL S+MTPA-CI or R-MTPA-Cl. The reaction was left for 2 hrs at room temperature, and then dried in N2. Analytical TLC plate was used to purify the bis-MTPA esters, to obtain 16.9 mg of 5-Ns MTPA ester (from R-MTPA-CI) and 16.0 mg of R-bis MTPA ester (from S+MTPA-CI).

### NMR Analysis

Two sets of samples were received for NMR analysis. The first set of samples were mono-esterified at position 12 of the molecule with the R (−) and S (+) diastereomers of Mosher's reagent. The second set of samples were di-esterified at positions 12 and 13 of the compound with either the R (−) diastereomer of Mosher's reagent at both positions or the S (+) diastereomer of Mosher's reagent at both positions.

Initially 1D-¹H spectra were run on each sample with 2D-spectra run to confirm the shifts of the resonances that had shifted due to the addition of the Mosher's reagents.

All spectra were run on a Bruker Avance III NMR spectrometer, with a 16.4T magnet operating at 700 MHz proton frequency, equipped with a cryogenically cooled 5 mm probe. The 1D-¹H spectra were acquired into 65536 complex points using a 30° pulse over a 20 ppm sweep width.

The 1D time domain spectra were Fourier transformed into 64K real points after apodization with a decaying exponential with a line broadening factor of 0.3 Hz. The spectra were then phased to pure absorption, baseline corrected and referenced to the residual CHCl₃ resonance at 7.26 ppm. 2D-spectra acquired were ¹H-COSY, ¹H-NOESY and ¹H-¹³C-HSQC.

### Results and Discussion

There are 3 chiral carbons that have to be resolved in the structure of compound (III). The stereochemistry of C-9 position was determined this time based on the optical rotation and comparison of data from literature. The specific rotation data we obtained on NC77(2)-44-46 were [a]D 20=−40.9 (c 0.30, CHCl₃) and [a]D 20=−46.9 (c 0.18, MeOH). In comparison to mueggelone (IV), a similar 10-membered lactone isolated from fresh water cyanobaterium Aphanizomenon flos-aquae and its total synthetic version as well as the synthetic diastereomer (Papendorf et al., 1997 and Ishigami et al., 2000), the stereochemistry of C-9 for compound (I) was suggested to be as S configuration. This is in agreement with the C-9 stereochemistry reported for another 10-member ring lactone phoracantholide I (V) isolated from metasternal gland of Phorancantha synonyma and its synthetic diastereomer (Kitahara et al., 1983). In this case, the C-9 R configuration is the same as S configuration as in compound (I), as the C-10,11 double bond has reversed the priority order:

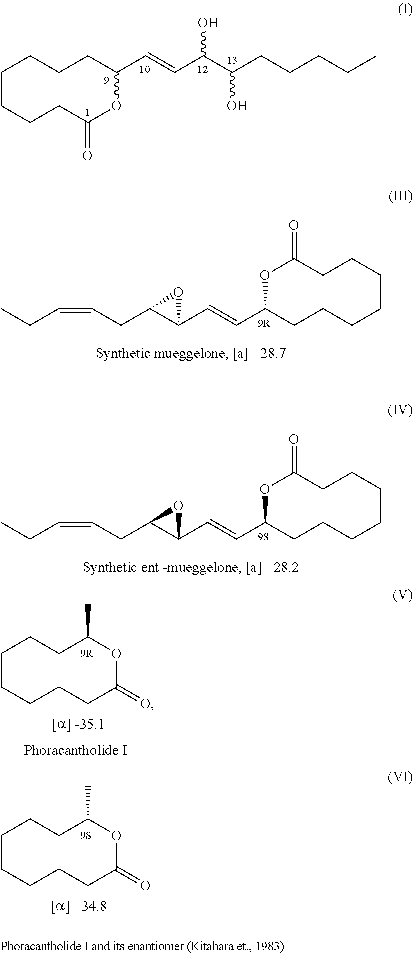

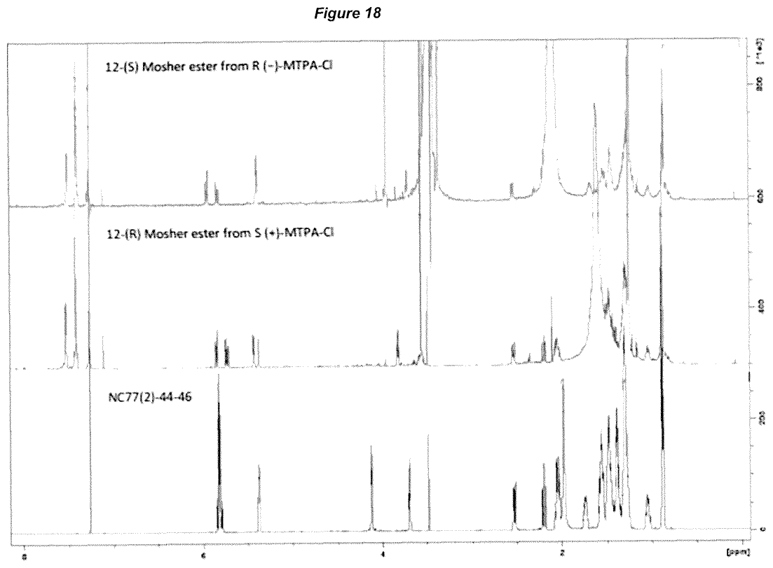

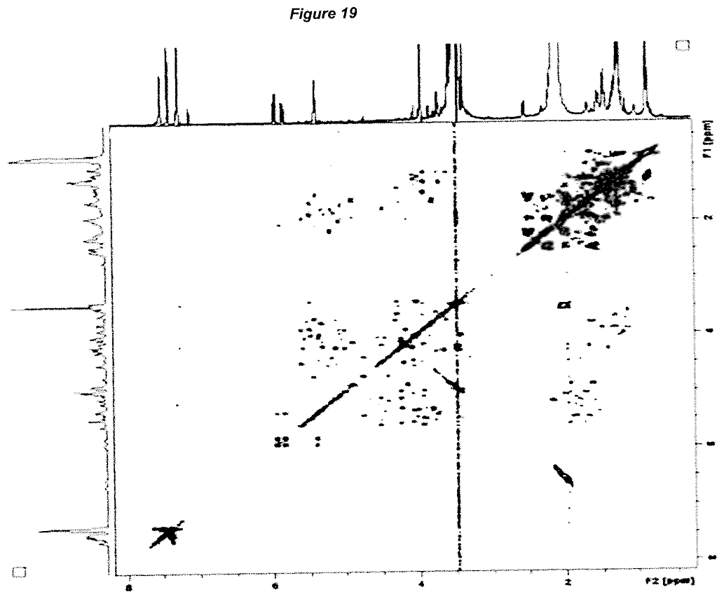

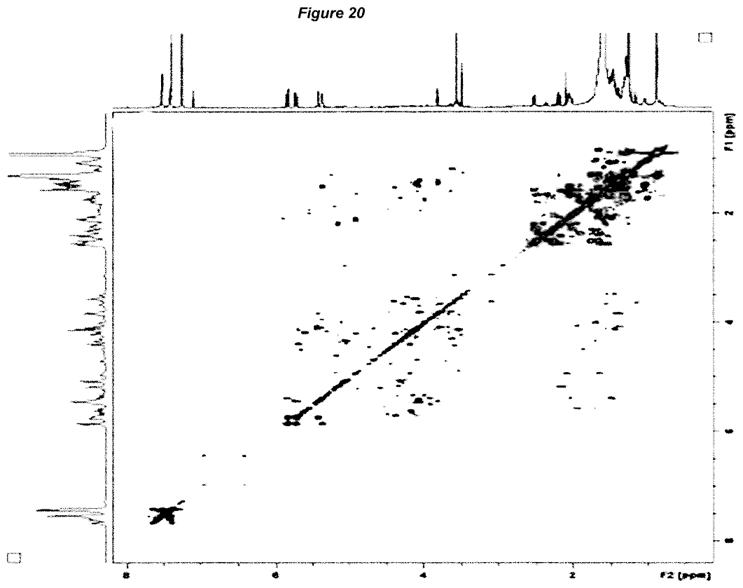

The absolute configuration for the C-12,13 diol was determined by using Mosher ester method (Hoye et al., 2007). The first attempt, using undried chloroform and pyridine yielded a pair of mono Mosher ester where only the C-12 hydroxyl group was esterified (¹H-NMR and COSY spectra shown in FIGS. 18-20).

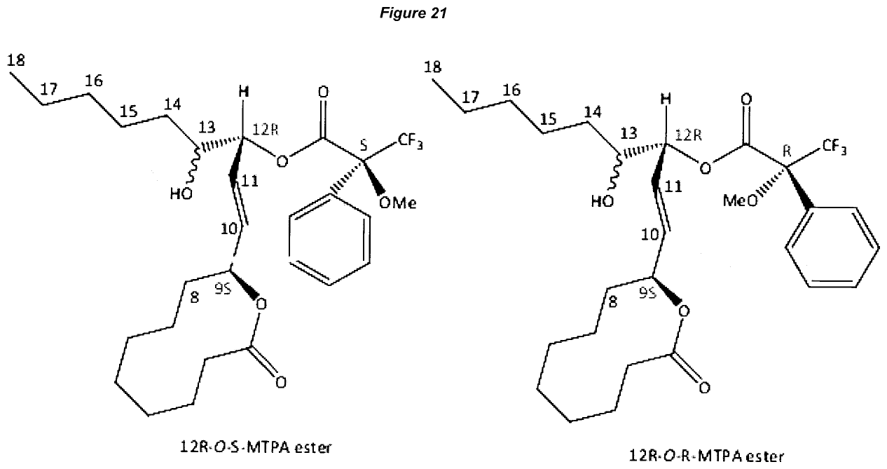

The differences of chemical shift for protons in S and R Mosher esters are listed in Table 14. Clearly, the phenyl ring shielding effect was on protons of C13 to C18 for the S-MTPA ester, and on C8 to C11 for the R-MTPA ester (FIG. 21). According to the Mosher's rule, the absolute configuration of C-12 was determined to be R.

TABLE 14
Table 1. δ (= δ_{S} − δ_{R}) data for
the 12-S- and R-MTPA Mosher esters

| Proton | δ S-ester (ppm) | δ R-ester (ppm) | Δδ^{SR} (= δ_{S} − δ_{R}) |
|---|---|---|---|
| H-11 | 5.83 | 5.73 | 0.10 |
| H-10 | 5.94 | 5.84 | 0.10 |
| H-8b | 1.53 | 1.49 | 0.04 |
| H-9 | 5.39 | 5.37 | 0.02 |
| H-15a | 1.53 | 1.55 | −0.02 |
| H-18 | 0.86 | 0.89 | −0.03 |
| H-15b, 16, 17 | 1.25 | 1.29 | −0.04 |
| H-12 | 5.38 | 5.43 | −0.05 |
| H-14 | 1.31 | 1.42 | −0.11 |
| H-13 | 3.71 | 3.82 | −0.11 |

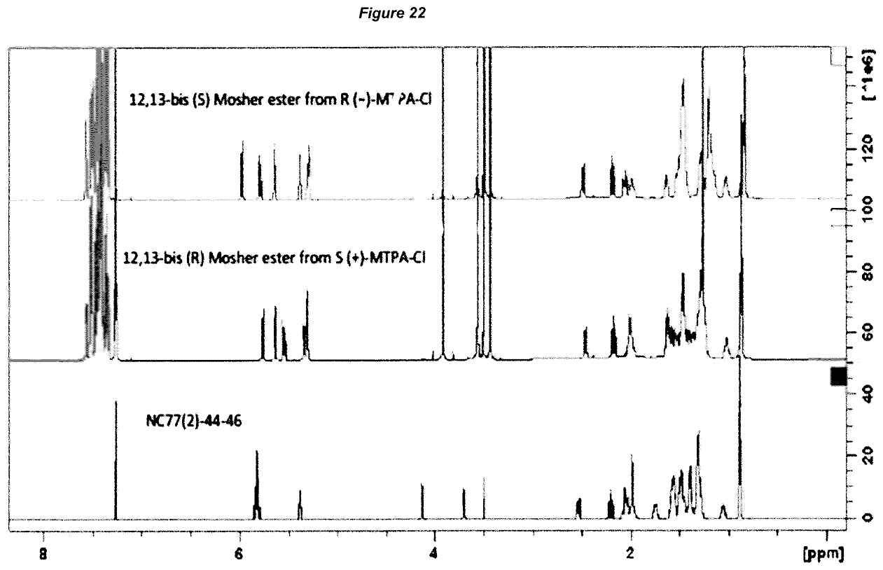

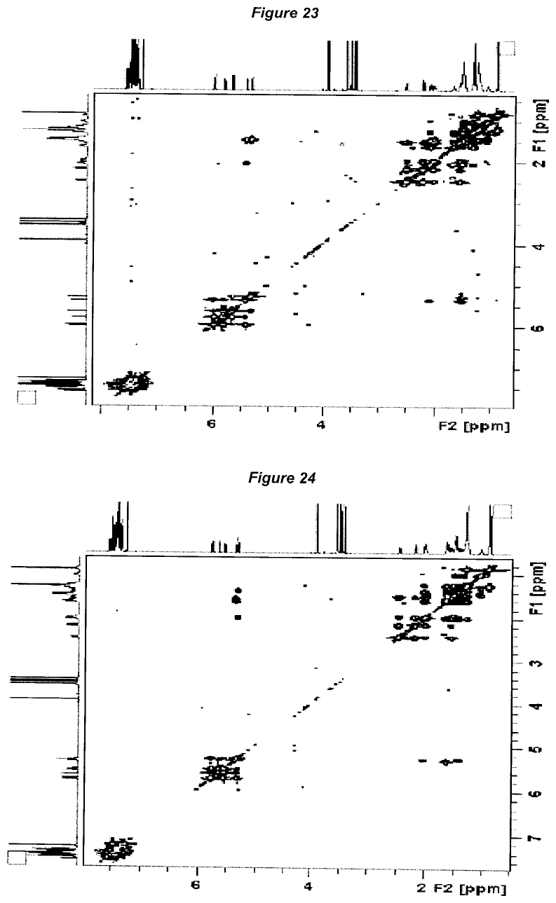

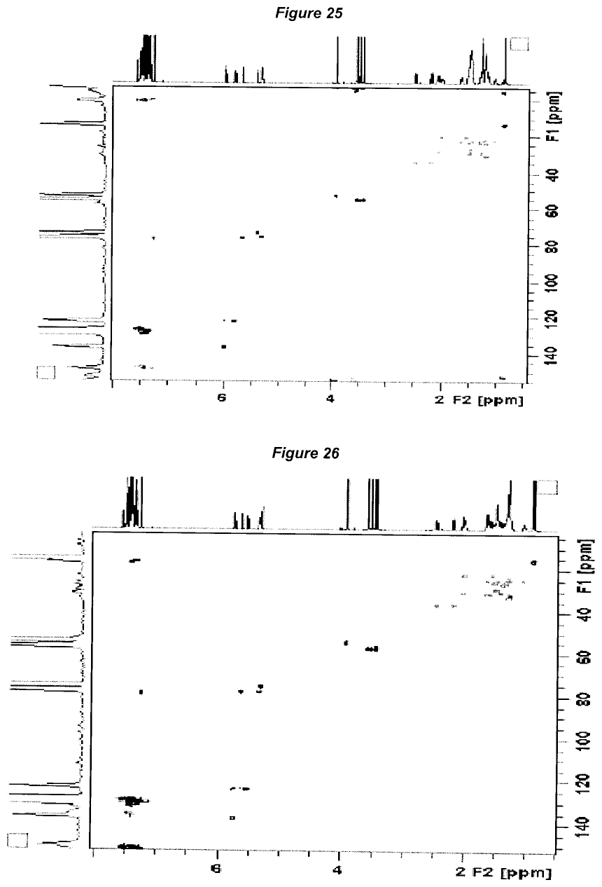

The Mosher ester preparation was repeated by using molecular sieve treated chloroform and pyridine, and a R/S pair of C-12,13-bis Mosher esters were obtained. Proton NMR of the R/S pair MTPA esters and their COSY and HSQC spectra are shown in FIGS. 22-26.

The proton assignment in the pair of 12,13-bis MTPA esters was accomplished by analyzing the COSY and HSQC spectra. The chemical shift differences between the S and R 12,13-bis-MTPA esters were listed in Table 15.

TABLE 15
Table 2. δ (= δ_{S} − δ_{R}) data for 12,13-
bis S- and R-MTPA Mosher esters

| Proton | δ S-ester (ppm) | δ R-ester (ppm) | Δδ^{SR} (= δ_{S} − δ_{R}) |
|---|---|---|---|
| H-11 | 5.78 | 5.54 | 0.25 |
| H-10 | 5.97 | 5.75 | 0.21 |
| H-8b | 1.48 | 1.40 | 0.08 |
| H-9 | 5.38 | 5.30 | 0.07 |
| H-8a | 2.04 | 1.99 | 0.05 |
| H-12 | 5.63 | 5.63 | 0.00 |
| H-15a | 1.46 | 1.60 | −0.02 |
| H-18 | 0.83 | 0.87 | −0.03 |
| H-15b | 1.18 | 1.26 | −0.04 |
| H-13 | 5.29 | 5.34 | −0.05 |
| H-17 | 1.20 | 1.26 | −0.06 |
| H-16 | 1.19 | 1.26 | −0.06 |
| H-14 | 1.46 | 1.60 | −0.13 |

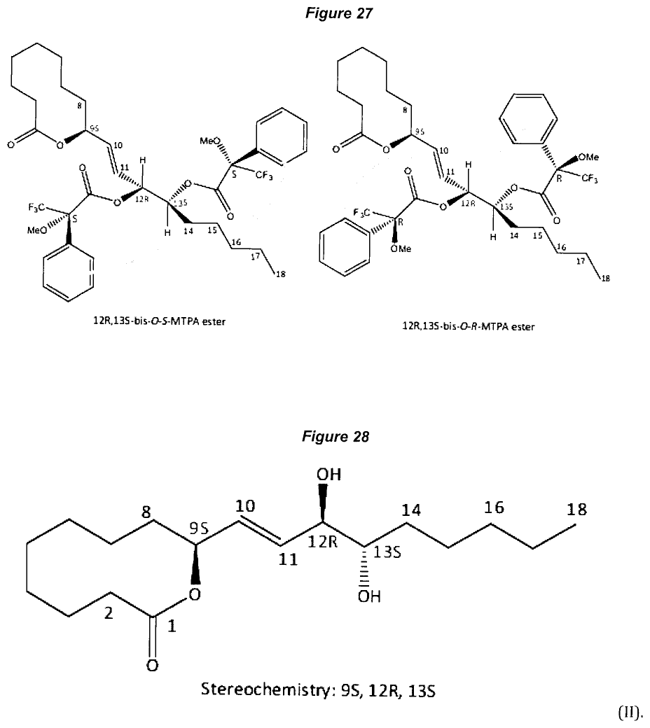

Based on these data and the approach proposed by Freire and co-workers (2005), the absolute configuration of the diols (C12 and C13) was assigned as R and S, respectively. The phenyl shielding effects on some of the protons for the two Mosher esters are shown in FIG. 27.

As such, the stereochemistry of compound (II) was defined to be in 9S, 12R, and 13S configuration:

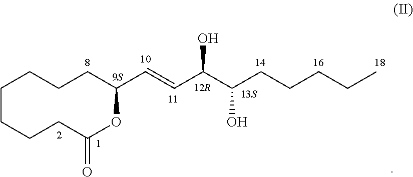 [SMILES: CCCCC[C@H](O)[C@H](O)/C=C/[C@@H]1CCCCCCCC(=O)O1]

### Example 10. In Vivo Testing of Pure Compound (II) in a Murine Flank Tumor Model

### Establishing of the Tumor Model:

Animal studies were conducted under ACC protocol #17-020 and Biosafety protocol #6007077 approved by the Atlantic Veterinary College (AVC, UPEI). Briefly, homozygous outbred nude mice (strain J:NU, 4-weeks old, female) suitable as immunodeficient tumor transplant hosts were procured from a commercial supplier (Jackson Labs, Maine, USA) and acclimatized in a barrier facility (AVC, UPEI) for one week.

To establish the tumors, MDA-MB-231 human breast cancer cells were cultured in vitro and harvested when they were between 60 to 80% confluent. Under anesthetized conditions, each the mouse was injected subcutaneously into both right and left flanks with 107 cells/100 μl (50:50 in matrigel:DMEM). Tumors development was monitored over a 2-week period. At 2 weeks, tumor established mice (tumor volume ˜20 mm³) were selected and randomly grouped based on the tumor volume and body weight into ten mice per group as follows:
Group 1: Vehicle control (VC, DMSO 2%, Kholliphor EL 2%)
Group 2: Compound (II), 10 mg/kg body weight
Group 3: Compound (II), 5 mg/kg body weight

### Evaluation of Pure Compound (II):

Seaweed extract was dissolved in molecular grade DMSO, liquid emulsion was prepared in Kholliphor EL (Sigma, USA) and the final injectable solution was prepared using sterile normal saline.

Mice with established tumors were treated every second day by intraperitoneal injections of test extracts over a 4-week time course. From each group a minimum of 16 tumors were selected for analysis. Tumors were measured weekly using external calipers and volumes established using the modified ellipsoidal formula: tumor volume=0.5 (length×width²).

In addition, body weights and general animal health were assessed twice weekly during the treatment period. At study termination, mice were sacrificed, and tumors excised from the flank. To evaluate tumor metastasis, an explorative necropsy was conducted in each mouse and the presence and frequency of additional tumors at sites distal to the flank were assessed. Tumor tissue from the flank region was collected and preserved in 10% neutral buffered formalin for histology. Variance within each of the treatment groups to vehicle control was assessed over the four-week time course by one-way ANOVA.

### Results

### Tumor Volumes

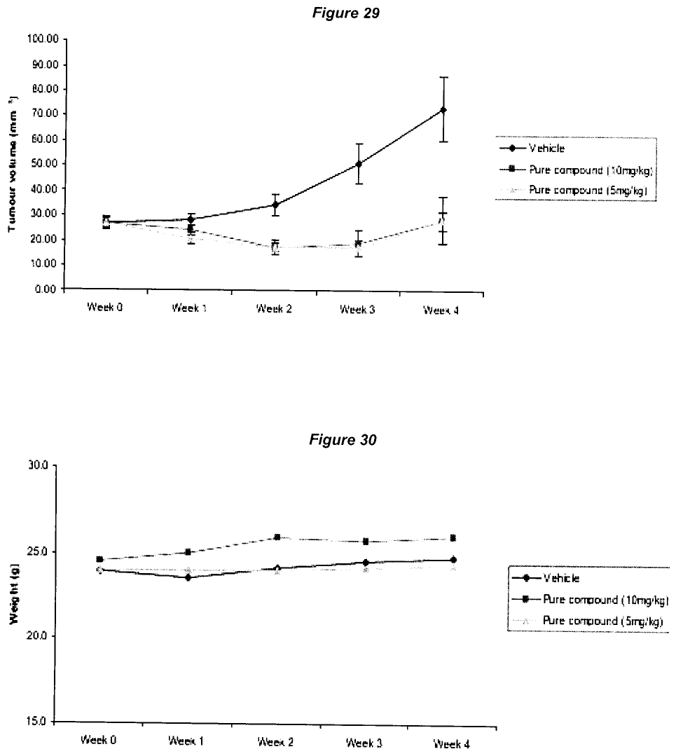

Over the course of the study, tumor volumes in control groups increased by approximately 2.5-fold, from 26 mm³ to 73 mm³ (Table 16, FIG. 29). In contrast, no significant change in tumor size was observed in mice treated with the pure compound at both tested concentrations during the 4-week study. On an average at the end of 4-weeks, tumors in pure compound-treated animals at 5 and 10 mg/kg remained close to their starting volume, 27 mm³. This profile of tumors failing to progress in size suggest that the pure compound exerted an antiproliferative effect.

TABLE 16
Tumor volumes over the four-week time course.

| | Concentration | # of | Tumour volumes (mm³) ± SEM | | | | |
|---|---|---|---|---|---|---|---|
| Group | (mg/kg b. wt.) | tumours | Week 0 | Week 1 | Week 2 | Week 3 | Week 4 |
| Vehicle | 0 | 18 | 26.78 ± 2.56 | 28.03 ± 2.23 | 34.43 ± 4.52 | 51.00 ± 8.03 | 73.28 ± 12.90 |
| Pure | 10 | 18 | 27.09 ± 1.60 | 24.06 ± 1.98 | 17.40 ± 2.88*¶ | 18.95 ± 5.34*¶ | 28.37 ± 9.37*¶ |
| Compound | 5 | 18 | 26.94 ± 1.98 | 20.83 ± 2.20 | 17.38 ± 2.04*¶ | 17.23 ± 3.18*¶ | 28.01 ± 3.61*¶ |

ANOVA, variance in tumor volume within each group when compared to the vehicle group:
*Significant at P ≤ 0.05,
¶Significant at P ≤ 0.01

### Body Weights:

Over the study course, the body weights of mice in all treatment groups remained stable with no adverse effects observed (FIG. 30). The average starting weight at acclimatization was around 22 g and average weight at study end was around 25 g.

### General Animal Health

Throughout the study course, all animals were found in good general health conditions except one animal that showed light patchy skin discoloration around the neck and body at 2-week treatment period. The animal was immediately segregated and excluded from the treatment. However, upon close monitoring the other general health conditions, in the segregated animal, such as eating, behavior and body weight gain was found to be normal over the rest of the study period. Therefore, the observed skin change was considered incidental or background related but not infectious or treatment related.

### Necropsy

Grossly, no obvious abnormalities related to distant tumor metastasis were detected in any of the animals in this study. Internal organs, with high potential for MDA-MB-231 cell metastasis, such as liver, lungs, axillary lymph nodes and diaphragm looked normal.

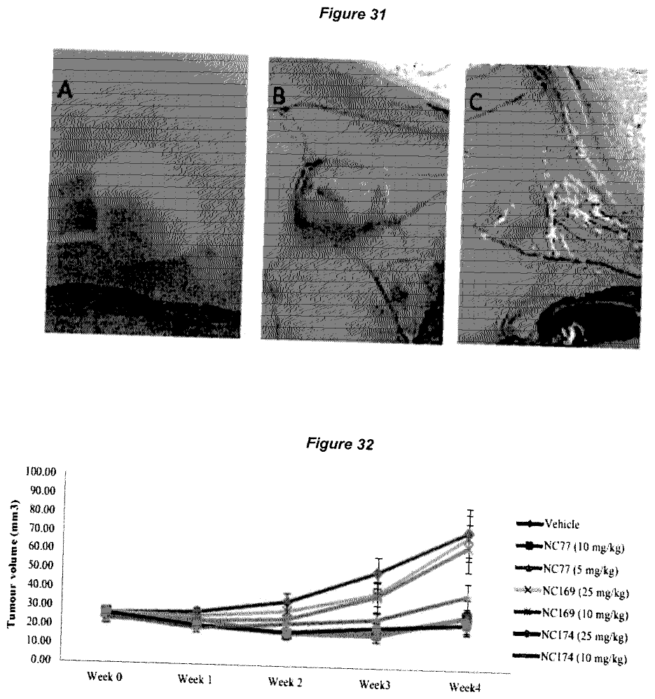

Representative images of tumors at necropsy are shown in FIG. 31 with extracted tumor weights summarized in Table 17.

TABLE 17
Extracted tumor weights at necropsy.

| | Concentration | # of | | Tumour weight |
|---|---|---|---|---|
| Group | (mg/kg b.wt.) | tumours | [symbol] | (mg) ± SEM |
| Vehicle | 0 | 18 | [symbol] | 58.35 ± 16.84 |
| Pure | 10 | 18 | [symbol] | 27.29 ± 8.07 |
| Compound | 5 | 18 | [symbol] | 34.64 ± 15.78 |

Note:
Extracted weights exhibit far more variability (excess tissue, blood, etc) than volumes that precludes statistical analysis. [symbol]
indicates data missing or illegible when filed

### Example 11. In Vitro and In Vivo Testing of Synthesized Compound (II)

Background: A synthesis regimen was developed to produce the compound of formula (II). The current report outlines the result of the study to evaluate the activity of this synthesized compound in in vitro and in vivo models.

### In Vitro Anticancer Evaluation of the Test Compound

Experimental: Stock solutions of the test compounds were prepared in dimethylsulfoxide (DMSO) at 10 mg/ml and stored at −20° C. until used. This preparation ensures that the DMSO content delivered to cells in culture never exceeds 1%. Appropriate cells number, based on previous assays, were seeded in 96 well plate and incubated with test compounds (up to 8 log concentrations, 0 to 50 or 100 μg/ml) for 72 h. Cell proliferation was initially assessed using a standard colorimetric indicator of metabolic activity (CIMA) assay. In this assay, reduction of yellow tetrazolium salt (MTT) to purple formazan by mitochondrial reductases enzymes in viable cells were measured as change in absorbance (λ=500-600).

Seven human cell lines were evaluated: U373 (glioblastoma-astrocytoma), A549 (lung carcinoma), PC3 (prostate adenocarcinoma), THP1 (acute monocytic leukemia), MDA-MB-231 (mammary gland adenocarcinoma), SKOV3 (ovarian adenocarcinoma) and CCD1079Sk (normal skin fibroblast) cells.

Results: The synthetic isomer of lead compound (II): 12R, 13S-Dihydroxy-10-Octadecen-9S-olide (DHODO) was tested in tumour cell lines and found to exhibit similar profiles of broad spectrum antiproliferative activity (Table 18). The activity of the synthetic compound was as good as, or better than that observed in earlier studies with NC-77 extracted as a pure compound (II).

TABLE 18
Summary of in vitro testing

| Human cells | DHODO (IC₅₀) |
|---|---|
| A549 | 22.03 |
| MDA-MB-231 | 34.26 |
| PC3 | 37.66 |
| U373 | 39.15 |
| THP-1 | 25.87 |
| SKOV3 | 67.66 |
| 1079SK | 34.86 |

### In Vivo Anticancer Evaluation of Synthesized Compound (II)

Test material was dissolved in DMSO and the final injectable solution was prepared using PBS. Homozygous outbred nude mice (strain J:NU, 6-weeks old, female) suitable as immune-deficient tumour transplant hosts will be procured from a commercial supplier (Jackson Labs, Maine, USA) and acclimatized in a barrier facility (AVC) for one week. Mice were weighed and random groups of ten mice were established based on the tumour volume and body weight as follows:

1. Group 3: DHODO, 1 mg/kg

2. Group 4: DHODO, 5 mg/kg

3. Group 5 DHODO, 10 mg/kg

4. Group 6: Vehicle control

To establish the tumours, MDA-MB-231 breast cancer cells (2.5 million cells in 100 pl 50:50 matrigel:DMEM) were injected subcutaneously into both right and left flanks. Tumours were evaluated over a 2-week period until sufficient volume growth (approximately 20 mm3) for subsequent analysis had occurred. During this period, body weights were measured twice weekly.

Mice with established tumours were treated every second day by intraperitoneal injections of test compounds over a 4-week time course. From each group a minimum of 15 tumours were selected for analysis. Tumours were measured weekly using external calipers and volumes established using the modified ellipsoidal formula: tumour volume=0.5 (length×width2). In addition, body weights and general animal health were assessed twice weekly during the treatment period. At study termination, mice were sacrificed and tumours excised from the flank was weighed. To evaluate tumour metastasis, an explorative necropsy was conducted in each mouse and the presence and frequency of additional tumours at sites distal to the flank were assessed.

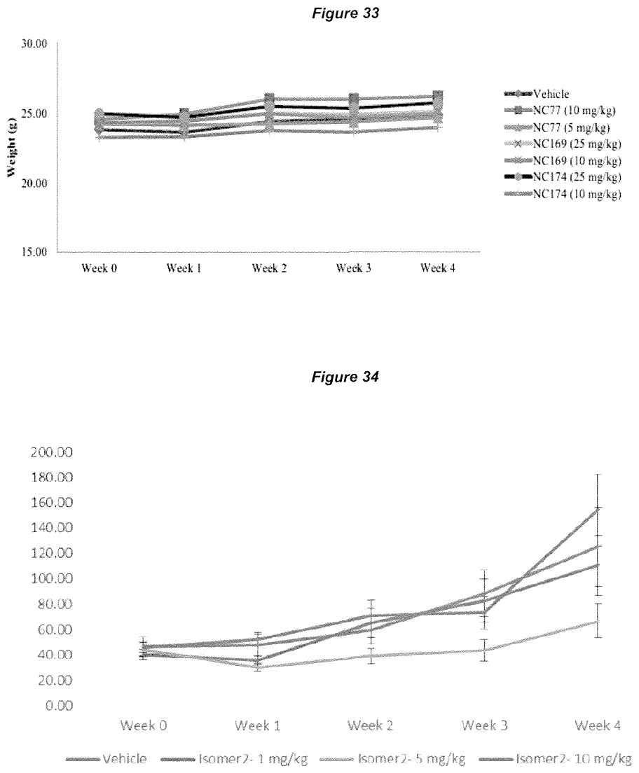

Results: Due to prolonged periods of extreme cold temperatures prohibiting transport of live animals, mice were retained by the air transport company for ten days rather than the standard two to three-day period. No overt issues with general animal health were observed during the one week acclimation period and throughout the study period. However, animals did not gain weight in a typical fashion (Table 19 and FIG. 33) and exhibited average gains of less than one gram compared to the expected gain of at least four grams.

TABLE 19
Body weight changes over the four-week trial

[symbol]

| Body weight (g) | Week 1 | Week 2 | Week 3 | Week 4 | Δ |
|---|---|---|---|---|---|
| DMSO vehicle | 22.8 | 23.3 | 22.7 | 23.3 | 0.5 |
| DHODO, 1 mg/kg | 21.9 | 22.5 | 22.1 | 22.5 | 0.6 |
| DHODO, 5 mg/kg | 23.8 | 24.8 | 24.3 | 24.9 | 0.9 |
| DHODO, 10 mg/kg | 24.2 | 24.8 | 24.3 | 25.0 | 0.8 |

[symbol]

indicates data missing or illegible when filed

Tumour growth profiles also showed an atypical pattern compared to previous studies. Tumours entered their exponential growth phase in many of the test groups much more rapidly (two weeks in the current trial vs the more than four weeks standard). This effect was especially evident for synthesized compound (II)-treated groups at 1 and 10 mg/kg (Table 20) and resulted in much larger final tumour volumes and a high degree of variability in size between tumours. The exponential growth phase represents a lethal stage of tumour development at which an external intervention would be highly unlikely to have a beneficial effect.

It is not clear whether the lack of body weight gains and early exponential growth are related two each other or stress arising from the prolonged transport period. However, these observations should be taken into account when interpreting the results of the current study.

TABLE 20
Tumour volumes at necropsy

| Treatment | Tumour weight (g) | SEM |
|---|---|---|
| Vehicle | 0.0623 | 0.0140 |
| DHODO, 1 mg | 0.0815 | 0.0215 |
| DHODO, 5 mg | 0.0380 | 0.0086 |
| DHODO, 10 mg | 0.0913 | 0.0155 |

No obvious gross abnormalities related to distant tumour metastasis were detected in any of the animals. Internal organs, with high potential for MDA-MB-231 cell metastasis, such as liver, lungs, axillary lymph nodes and diaphragm looked normal. In terms of tumour volumes during the course of the study (Table 21, FIGS. 32 and 34), several observations were made in a biological context:
1. Tumour volumes were reduced by 50% in animals treated with Synthesized compound (II) at 5 mg/kg. Exponential tumour growth was not reached in this group until near the end of the study period.
2. No reduction in tumour growth was observed with animals treated with Synthesized compound (II) at 1 and 10 mg/kg. As noted above, exponential tumour growth was reached in these two groups early in the study period.
3. The large degree of variability in tumour sizes prohibits a conclusion on the statistical relevance of the in vivo findings. However, biological trends support the conclusion that the synthesized compound (II) compound exhibits anti-proliferative activity.

TABLE 21
Tumour size expressed in volume (mm³) over the four-week time course

| | Week 0 | | Week 1 | | Week 2 | | Week 3 | | Week 4 | |
|---|---|---|---|---|---|---|---|---|---|---|
| Treatment | Size | SEM | Size | SEM | Size | SEM | Size | SEM | Size | SEM |
| Vehicle | 46.69 | 7.81 | 48.29 | 8.62 | 59.88 | 11.64 | 88.39 | 18.02 | 124.90 | 30.98 |
| DHODO, 1 mg/kg | 40.59 | 3.94 | 36.34 | 3.47 | 65.15 | 11.25 | 82.66 | 17.01 | 110.23 | 23.29 |
| DHODO, 5 mg/kg | 44.24 | 4.39 | 30.47 | 3.18 | 39.17 | 6.11 | 43.73 | 8.50 | 66.83 | 13.38 |
| DHODO, 10 mg/kg | 46.33 | 4.18 | 52.86 | 5.47 | 71.23 | 11.35 | 73.39 | 12.69 | 154.29 | 28.54 |

Summary: Synthesized compound II (DHODO) significantly reduced tumour cell viability and growth in vitro across a spectrum of established cell types.

Sufficient red flags related to animal stress levels and tumour properties were raised in relation to the in vivo trial to suggest that conclusions should be tempered. The results with DHODO at 1 and 10 mg/kg are limited by the early onset exponential tumour growth and lack of weight gain that occurred in the study.

DHODO at 5 mg/kg inhibited tumour growth consistent with its non-synthetic counterpart. Taken with the in vitro results and the analytical characterization of the synthetic compound, it is reasonable to conclude that synthetic compound is the same as the purified extracted version and possess similar activity. DHODO would not be expected to impact tumour size once exponential growth was entered.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

All publications and patent applications cited in this specification are herein incorporated by reference as if each individual publication or patent application were specifically and individually indicated to be incorporated by reference.

### REFERENCES

Freire, F., Seco, J. M., Quinoa, E. and Riguera, R. Determining the absolute stereochemistry of secondary/secondary diols by 1H-NMR: basis and applications, J. Org. Chem. 2005, 70, 3778-3790.
Hoye, T. R., Jeffrey, C. S. and Shao, F. Mosher ester analysis for the determination of absolute configuration of stereogenic (chiral) carbinol carbons, Nature Protocols 2007, 2, 2451-2458.
Ishigami, K., Motoyoshi, H., and Kitahara, T. First total synthesis of mueggelone, Tetrahedron Lett. 2000, 8897-8901.
Kitahara, T., Koseki, K., and Mori, K. The synthesis and the absolute configuration of phoracantholide I and J; the defensive secretion of the eucarypt longicorn, Phoracantha synonyma, Agric. Biol. Chem. 1983, 47, 389-393.
Papendorf, O., Konig, G. M., Wright, A. D., Chorus, I. and Oberemm, A. Mueggelone, a novel inhinitor of fish development from the fresh water cyanobacterium Aphanizomenon flos-aquae, J. Nat. Prod. 1997, 60, 1298-1300.

1. A purified compound having the formula (II):

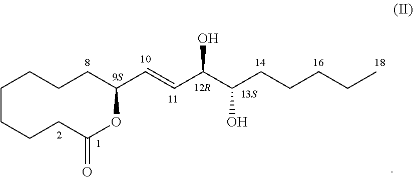 [SMILES: CCCCC[C@H](O)[C@H](O)/C=C/[C@@H]1CCCCCCCC(=O)O1]

2-19. (canceled)

20. The compound of claim 1, in a racemic mixture, wherein said compound is enriched compared to another stereoisomer thereof.

21. The compound of claim 1, incorporated into a formulation for oral or parenteral use.

22. The compound of claim 21, wherein said oral formulation is a nutraceutical or nutritional formulation.

23. A method for the treatment of cancer, comprising administering a growth-inhibiting concentration of the compound of claim 1, to a mammal in need thereof.

24. The method of claim 23, wherein the mammal is a human.

25. The method of claim 23, wherein the cancer is the cancer is selected from the group consisting of: brain, lung, prostate, blood, breast, skin, and ovarian cancers.

26. A composition comprising the compound of claim 1, in admixture with a physiologically-acceptable excipient.

27. The composition of claim 26, wherein the compound is substantially stereoisomerically pure.

28. The composition of claim 26, wherein said excipient is acceptable for oral or parenteral administration

29. The composition of claim 26, in combination with one or more other therapeutic agent.

30. The composition of claim 29, wherein said other therapeutic agent is an anti-cancer agent.

31. The composition of claim 26, incorporated into a formulation for oral or parenteral use.

32. A method for the treatment of cancer, comprising administering a growth-inhibiting concentration of the composition of claim 26, to a mammal in need thereof.

33. The method of claim 32, wherein the mammal is a human.

34. The method of claim 32, wherein the cancer is the cancer is selected from the group consisting of: brain, lung, prostate, blood, breast, skin, and ovarian cancers.
